SEQUENCE LISTING (49 sequences)

SEQ ID NO: 1 - DNA, 26 bp, Artificial Sequence
  Description of Artificial Sequence; note = synthetic construct
gagcctggaattcataggcgctttgc

SEQ ID NO: 2 - DNA, 25 bp, Artificial Sequence
  Description of Artificial Sequence; note = synthetic construct
ttttctggatccatgattcctctcc

SEQ ID NO: 3 - DNA, 34 bp, Artificial Sequence
  Description of Artificial Sequence; note = synthetic construct
cgagaattcataatgaaacgaaccgtcactatag

SEQ ID NO: 4 - DNA, 34 bp, Artificial Sequence
  Description of Artificial Sequence; note = synthetic construct
gtacttttttctttttcgttaacagcctaggtgc

SEQ ID NO: 5 - RNA, 59 nt, Artificial Sequence
  Description of Artificial Sequence; note = synthetic construct
gaauuccgugcaaugcacgggagagguucgcgaacucccucuauaaaaaacuauggaaa

SEQ ID NO: 6 - RNA, 74 nt, Artificial Sequence
  Description of Artificial Sequence; note = synthetic construct
guaaauauaaucuuguugcuuaaaaaaacgaauaacgugguucgaaaccaucccacguaaaaaaacuaaggaga

SEQ ID NO: 7 - RNA, 58 nt, Artificial Sequence
  Description of Artificial Sequence; note = synthetic construct
aaauuccgugcaccgcgcgggagagguucgcgaacucccucuauaaaaacuauggaaa

SEQ ID NO: 8 - RNA, 73 nt, Artificial Sequence
  Description of Artificial Sequence; note = synthetic construct
guaaaauauaaucuuguugcuuaaaaaacgaauaacgugguucgaaaccaucccacguaaaaaacuaaggaga

SEQ ID NO: 9 - RNA, 61 nt, Artificial Sequence
  Description of Artificial Sequence; note = synthetic construct
gaauuucacagcuuuucugugagagagguucgcgaacucccucuauaaaaaacuaaggcaa

SEQ ID NO: 10 - RNA, 79 nt, Artificial Sequence
  Description of Artificial Sequence; note = synthetic construct
uuaauuaacuaacuguuuaauguuuucuuguaauaagacaguucgcaauccauccugucuauaaacaaaaccagggcaa

SEQ ID NO: 11 - RNA, 79 nt, Artificial Sequence
  Description of Artificial Sequence; note = synthetic construct
uauauauuauauaaugacuaggaaaaauaauauauaagacaguucgaaagccuccugucuuuaaauaaaacuacggugu

SEQ ID NO: 12 - RNA, 59 nt, Artificial Sequence
  Description of Artificial Sequence; note = synthetic construct
aaaauccaauguaauuugguagagguucguaaccaucccucuauaaaaaacuaagggcu

SEQ ID NO: 13 - RNA, 79 nt, Artificial Sequence
  Description of Artificial Sequence; note = synthetic construct
auaauaucauauuuuagaauauaaaaaaauaaauauggacaguucguaaccauccugucccuaaauaaaacuauggagg

SEQ ID NO: 14 - RNA, 57 nt, Artificial Sequence
  Description of Artificial Sequence; note = synthetic construct
aaaaucgagaauuucucggacugguucggaaacuucccagaauaaaaacuaaguauc

SEQ ID NO: 15 - RNA, 74 nt, Artificial Sequence
  Description of Artificial Sequence; note = synthetic construct
uuccauauuagaaugugaacuguugcaauuguaaccaagguucaucaaccaucccuuguaaaaaacucggagaa

SEQ ID NO: 16 - RNA, 61 nt, Artificial Sequence
  Description of Artificial Sequence; note = synthetic construct
uauaaucaagacgcucgucuuggacugguucggaaacuucccagaauaaaaacuaaguauc

SEQ ID NO: 17 - RNA, 73 nt, Artificial Sequence
  Description of Artificial Sequence; note = synthetic construct
ccuucugcuagugugaauaguugagacugugucuccggaguucguaaccuccuccgucacaaaacuaggaaug

SEQ ID NO: 18 - RNA, 76 nt, Artificial Sequence
  Description of Artificial Sequence; note = synthetic construct
auaugagaaaauagcauguuggcaaaauugccacacgugguucauucauaccaucccacguaaaaaacuaggagga

SEQ ID NO: 19 - RNA, 58 nt, Artificial Sequence
  Description of Artificial Sequence; note = synthetic construct
aaaaucggcaaucgugccggacugguucggaaaaucccagaaaaaaaaccaacgugug

SEQ ID NO: 20 - RNA, 75 nt, Artificial Sequence
  Description of Artificial Sequence; note = synthetic construct
acgaauuaauugauuguguuucugagacuuaauaauagcgguucgucaaccaucccgcuuaaaaaacuaggagau

SEQ ID NO: 21 - RNA, 75 nt, Artificial Sequence
  Description of Artificial Sequence; note = synthetic construct
cgugcuacgauuauauucgcuuuguuucaaucaacacgugguucguaaccaucccacguuaaaaaacuaggagga

SEQ ID NO: 22 - RNA, 73 nt, Artificial Sequence
  Description of Artificial Sequence; note = synthetic construct
caucacauacaguuugaccuaauuaggcgagcuggccugguucguaaacuucccaggauaaaaaccaagaacu

SEQ ID NO: 23 - RNA, 75 nt, Artificial Sequence
  Description of Artificial Sequence; note = synthetic construct
ccuucgauuauacuaaucuaaugugauuuuacucaccgugguucguaaccaucccacgcaaaaaaacuaggaagg

SEQ ID NO: 24 - RNA, 62 nt, Artificial Sequence
  Description of Artificial Sequence; note = synthetic construct
gaauucugcacuacugcaggagagguucgcgauuuaaaucccucuauaaaaaacuaaggaga

SEQ ID NO: 25 - RNA, 72 nt, Artificial Sequence
  Description of Artificial Sequence; note = synthetic construct
uaauaaaacacgugcauaaucgaguauuucucggacugguucgaaaacuucccagaauaaaaacuaagugac

SEQ ID NO: 26 - RNA, 62 nt, Artificial Sequence
  Description of Artificial Sequence; note = synthetic construct
ucgcagugagcaacaaaaugcucaccugggucgcaguaaccccaguuaacaaacaagggagg

SEQ ID NO: 27 - RNA, 73 nt, Artificial Sequence
  Description of Artificial Sequence; note = synthetic construct
cacagauaccgccgcuuauauuacaaucgccgcccgugguucgaaaaccucccacauuaaaaaacuaaggaaa

SEQ ID NO: 28 - RNA, 75 nt, Artificial Sequence
  Description of Artificial Sequence; note = synthetic construct
uugccuguauaauccgcaucuuuacuguccaacuucgcgguucgcaaaccucccgcguuaccaaaacuaggauuc

SEQ ID NO: 29 - RNA, 75 nt, Artificial Sequence
  Description of Artificial Sequence; note = synthetic construct
uugccuguauaauccgcgucuuuacuguccaacuucgcgguucgaaaaccucccgcgucaccaaaacuaggauuc

SEQ ID NO: 30 - RNA, 72 nt, Artificial Sequence
  Description of Artificial Sequence; note = synthetic construct
acagauaccgccgcuuauauuacaaucgccgccccgugguucgaaaaccucccacacuaaaaacuaaggaaa

SEQ ID NO: 31 - RNA, 74 nt, Artificial Sequence
  Description of Artificial Sequence; note = synthetic construct
ugcuaaaauagaacuuccccacuagaauaaccccccguaguucgcaaaccuccuacaauaaaaaacuagguaaa

SEQ ID NO: 32 - RNA, 95 nt, Artificial Sequence
  Description of Artificial Sequence; note = synthetic construct
aacgacguuuaaacaagccuacuguuuucuuuucuaagugguucguaacccucccacuugaacaacaacaauuguucgaaacaaaacuaggaaaa

SEQ ID NO: 33 - RNA, 59 nt, Artificial Sequence
  Description of Artificial Sequence; note = synthetic construct
aaaauccgugcgauaugcgggagagguucuacguacacccucuauaaaaacuaaggacg

SEQ ID NO: 34 - RNA, 62 nt, Artificial Sequence
  Description of Artificial Sequence; note = synthetic construct
caauuccguacgcuuguacgggagagguuucuagcaaaacccucuauaaaaaacuagggacg

SEQ ID NO: 35 - RNA, 64 nt, Artificial Sequence
  Description of Artificial Sequence; note = synthetic construct
uguacaacaaacauaauuuuguuagagguucuuagcuucaacccucuauaaaaaacuaaggaca

SEQ ID NO: 36 - RNA, 64 nt, Artificial Sequence
  Description of Artificial Sequence; note = synthetic construct
ucuaaguuccgugcaaaauaauaacagagguuccuagccgaaaccucuauaaaaacuagacaug

SEQ ID NO: 37 - RNA, 74 nt, Artificial Sequence
  Description of Artificial Sequence; note = synthetic construct
agaugaauuuuaaguaaaauaguauaguuaauuuuggcgguuucuaaacacccgcuuuaacaaaauuuaggagg

SEQ ID NO: 38 - RNA, 71 nt, Artificial Sequence
  Description of Artificial Sequence; note = synthetic construct
uauucuauucaucguacauaaaugaauaucagagguuucuagcugaaacccucuauaaaaaacuagacauu

SEQ ID NO: 39 - RNA, 74 nt, Artificial Sequence
  Description of Artificial Sequence; note = synthetic construct
caauacagagaauuguuaaagaaauuuuuaauaaguagaugugcuagcaaaaccaucuuuaaaaacuagacuug

SEQ ID NO: 40 - RNA, 72 nt, Artificial Sequence
  Description of Artificial Sequence; note = synthetic construct
uuuuaaacuuugugaaucagacuacccuuuaauuaauaaguaacuagacaaccuuauaaaaaacuaggagga

SEQ ID NO: 41 - RNA, 24 nt, Artificial Sequence
  Description of Artificial Sequence; note = synthetic construct
rguucrracyuccyaaaacyargr

SEQ ID NO: 42 - RNA, 29 nt, Artificial Sequence
  Description of Artificial Sequence; note = synthetic construct
agarguyauraccyacuaaaaaacuarrr

SEQ ID NO: 43 - RNA, 25 nt, Artificial Sequence
  Description of Artificial Sequence; note = synthetic construct
rguucrracyuccyaaaaacyargr

SEQ ID NO: 44 - RNA, 29 nt, Artificial Sequence
  Description of Artificial Sequence; note = synthetic construct
agarguyuaraccyucuaaaaaacuarrr

SEQ ID NO: 45 - RNA, 106 nt, Artificial Sequence
  Description of Artificial Sequence; note = synthetic construct
ggcagaagugaaaaaauugaagaaaauccgugcgauaugcgggagagguucuagcuacacccucuauaaaaaacuaaggacgagcuguauccuuggauacggccuu

SEQ ID NO: 46 - RNA, 51 nt, Artificial Sequence
  Description of Artificial Sequence; note = synthetic construct
ggccgugcgauaugcgggagagguucuagcuacacccucauaaaaaacuaa

SEQ ID NO: 47 - RNA, 36 nt, Artificial Sequence
  Description of Artificial Sequence; note = synthetic construct
ggagagguucuagcuacacccucuauaaaaaacuaa

SEQ ID NO: 48 - RNA, 81 nt, Artificial Sequence
  Description of Artificial Sequence; note = synthetic construct
ggccgugcgauaugcgggagagguucuagcuacacccucuauaaaaaacuaaggacgagcuguauccuuggauacggccuu

SEQ ID NO: 49 - RNA, 82 nt, Artificial Sequence
  Description of Artificial Sequence; note = synthetic construct
aauccgugcgauaugcgggagagguucuagcuacacccucuauaaaaaacuaaggacgagcuguauccuuggauacggccuu

(19) United States
(12) Patent Application Publication
(10) Pub. No.: US 2010/0152212 A1
(43) Pub. Date: Jun. 17, 2010

(54) PREQ1 RIBOSWITCHES AND METHODS AND COMPOSITIONS FOR USE OF AND WITH PREQ1 RIBOSWITCHES

(75) Inventors: Ronald R. Breaker, Guilford, CT (US); Jeffrey E. Barrick, Lansing, MI (US); Adam Roth, Guilford, CT (US); Wade Winkler, Dallas, TX (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: Yale University, New Haven, CT (US)

(21) Appl. No.: 12/532,538

(22) PCT Filed: Mar. 24, 2008
(86) PCT No.: PCT/US08/58050

§ 371 Date: Mar. 5, 2010

Related U.S. Application Data
(60) Provisional application No. 60/919,410, filed on Mar. 22, 2007.

(51) Int. Cl. A61K 31/519; C12N 15/63; C07H 21/02; C12Q 1/68; A61K 31/52; C12P 19/34; C12N 1/21; A61P 31/04

(52) U.S. Cl. 514/263.37; 435/320.1; 536/23.1; 435/6; 514/265.1; 435/91.1; 435/252.3

(57) ABSTRACT

The preQ1 riboswitch is a target for antibiotics and other small molecule therapies. The preQ1riboswitch and portions thereof can be used to regulate the expression or function of RNA molecules and other elements and molecules. The preQ1 riboswitch and portions thereof can be used in a variety of other methods to, for example, identify or detect compounds. Compounds can be used to stimulate, active, inhibit and/or inactivate the preQ1 riboswitch. The preQ1 riboswitch and portions thereof, both alone and in combination with other nucleic acids, can be used in a variety of constructs and RNA molecules and can be encoded by nucleic acids.

### CROSS-REFERENCE TO RELATED APPLICATIONS

This application claims benefit of U.S. Provisional Application No. 60/919,410, filed Mar. 22, 2007. U.S. Provisional Application No. 60/919,410, filed Mar. 22, 2007, is hereby incorporated herein by reference in its entirety.

### STATEMENT REGARDING FEDERALLY SPONSORED RESEARCH

This invention was made with government support under Grant Nos. R33 DK07027 and GM 068819 awarded by the NIH, and under Grant No. EIA 0323510 awarded by NSF. The government has certain rights in the invention.

### FIELD OF THE INVENTION

The disclosed invention is generally in the field of gene expression and specifically in the area of regulation of gene expression.

### BACKGROUND OF THE INVENTION

Precision genetic control is an essential feature of living systems, as cells must respond to a multitude of biochemical signals and environmental cues by varying genetic expression patterns. Most known mechanisms of genetic control involve the use of protein factors that sense chemical or physical stimuli and then modulate gene expression by selectively interacting with the relevant DNA or messenger RNA sequence. Proteins can adopt complex shapes and carry out a variety of functions that permit living systems to sense accurately their chemical and physical environments. Protein factors that respond to metabolites typically act by binding DNA to modulate transcription initiation (e.g. the lac repressor protein; Matthews, K. S., and Nichols, J. C., 1998, Prog. Nucleic Acids Res. Mol. Biol. 58, 127-164) or by binding RNA to control either transcription termination (e.g. the PyrR protein; Switzer, R. L., et al., 1999, Prog. Nucleic Acids Res. Mol. Biol. 62, 329-367) or translation (e.g. the TRAP protein; Babitzke, P., and Gollnick, P., 2001, J. Bacteriol. 183, 5795-5802). Protein factors respond to environmental stimuli by various mechanisms such as allosteric modulation or post-translational modification, and are adept at exploiting these mechanisms to serve as highly responsive genetic switches (e.g. see Ptashne, M., and Gann, A. (2002). Genes and Signals. Cold Spring Harbor Laboratory Press, Cold Spring Harbor, N.Y.).

In addition to the widespread participation of protein factors in genetic control, it is also known that RNA can take an active role in genetic regulation. Recent studies have begun to reveal the substantial role that small non-coding RNAs play in selectively targeting mRNAs for destruction, which results in down-regulation of gene expression (e.g. see Hannon, G. J. 2002, Nature 418, 244-251 and references therein). This process of RNA interference takes advantage of the ability of short RNAs to recognize the intended mRNA target selectively via Watson-Crick base complementation, after which the bound mRNAs are destroyed by the action of proteins. RNAs are ideal agents for molecular recognition in this system because it is far easier to generate new target-specific RNA factors through evolutionary processes than it would be to generate protein factors with novel but highly specific RNA binding sites.

Although proteins fulfill most requirements that biology has for enzyme, receptor and structural functions, RNA also can serve in these capacities. For example, RNA has sufficient structural plasticity to form numerous ribozyme domains (Cech & Golden, Building a catalytic active site using only RNA. In: The RNA World R. F. Gesteland, T. R. Cech, J. F. Atkins, eds., pp. 321-350 (1998); Breaker, In vitro selection of catalytic polynucleotides. Chem. Rev. 97, 371-390 (1997)) and receptor domains (Osborne & Ellington, Nucleic acid selection and the challenge of combinatorial chemistry. Chem. Rev. 97, 349-370 (1997); Hermann & Patel, Adaptive recognition by nucleic acid aptamers. Science 287, 820-825 (2000)) that exhibit considerable enzymatic power and precise molecular recognition. Furthermore, these activities can be combined to create allosteric ribozymes (Soukup & Breaker, Engineering precision RNA molecular switches. Proc. Natl. Acad. Sci. USA 96, 3584-3589 (1999); Seetharaman et al., Immobilized riboswitches for the analysis of complex chemical and biological mixtures. Nature Biotechnol. 19, 336-341 (2001)) that are selectively modulated by effector molecules.

Bacterial riboswitch RNAs are genetic control elements that are located primarily within the 5′-untranslated region (5″-UTR) of the main coding region of a particular mRNA. Structural probing studies (discussed further below) reveal that riboswitch elements are generally composed of two domains: a natural aptamer (T. Hermann, D. J. Patel, Science 2000, 287, 820; L. Gold, et al., Annual Review of Biochemistry 1995, 64, 763) that serves as the ligand-binding domain, and an ‘expression platform’ that interfaces with RNA elements that are involved in gene expression (e.g. Shine-Dalgarno (SD) elements; transcription terminator stems). What is needed in the art are methods and compositions that can be used to regulate preQ₁ riboswitches, as well as functional preQ₁ riboswitches.

### BRIEF SUMMARY OF THE INVENTION

Disclosed herein is a regulatable gene expression construct comprising a nucleic acid molecule encoding an RNA comprising a preQ₁-responsive riboswitch operably linked to a coding region, wherein the riboswitch regulates expression of the RNA, wherein the riboswitch and coding region are heterologous. The riboswitch can comprise an aptamer domain and an expression platform domain, wherein the aptamer domain and the expression platform domain are heterologous. The riboswitch can also comprise two or more aptamer domains and an expression platform domain, wherein at least one of the aptamer domains and the expression platform domain are heterologous. At least two of the aptamer domains can exhibit cooperative binding.

Also disclosed is a riboswitch, wherein the riboswitch is a non-natural derivative of a naturally-occurring preQ₁-responsive riboswitch. The riboswitch can comprise an aptamer domain and an expression platform domain, wherein the aptamer domain and the expression platform domain are heterologous. The riboswitch can further comprise one or more additional aptamer domains. At least two of the aptamer domains can exhibit cooperative binding. The riboswitch can be activated by a trigger molecule, wherein the riboswitch produces a signal when activated by the trigger molecule.

Further disclosed is a method of detecting a compound of interest, the method comprising: bringing into contact a sample and a riboswitch, wherein the riboswitch is activated by the compound of interest, wherein the riboswitch produces a signal when activated by the compound of interest, wherein the riboswitch produces a signal when the sample contains the compound of interest, wherein the riboswitch comprises a preQ₁-responsive riboswitch or a derivative of a preQ₁-responsive riboswitch. The riboswitch can change conformation when activated by the compound of interest, wherein the change in conformation produces a signal via a conformation dependent label. The riboswitch can also change conformation when activated by the compound of interest, wherein the change in conformation causes a change in expression of an RNA linked to the riboswitch, wherein the change in expression produces a signal. The signal can be produced by a reporter protein expressed from the RNA linked to the riboswitch.

Also disclosed is a method comprising: (a) testing a compound for inhibition of gene expression of a gene encoding an RNA comprising a riboswitch, wherein the inhibition is via the riboswitch, wherein the riboswitch comprises a preQ₁-responsive riboswitch or a derivative of a preQ₁-responsive riboswitch, (b) inhibiting gene expression by bringing into contact a cell and a compound that inhibited gene expression in step (a), wherein the cell comprises a gene encoding an RNA comprising a riboswitch, wherein the compound inhibits expression of the gene by binding to the riboswitch.

Disclosed is a method of identifying preQ₁-responsive riboswitches, the method comprising assessing in-line spontaneous cleavage of an RNA molecule in the presence and absence of preQ₁, wherein the RNA molecule is encoded by a gene regulated by preQ₁, wherein a change in the pattern of in-line spontaneous cleavage of the RNA molecule indicates a preQ₁-responsive riboswitch.

Also disclosed is a method of inhibiting gene expression, the method comprising bringing into contact a compound and a cell, wherein the compound has the structure of Formula I:

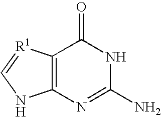

where R¹ is CH, N, C—NH₂, C—CH₂—NH₂, C—CN, C—C(O)NH₂, C—CH═NH, C—CH₂—N(CH₃)₂, or C-hydrogen bond donor, wherein the cell comprises a gene encoding an RNA comprising a preQ₁-responsive riboswitch, wherein the compound inhibits expression of the gene by binding to the preQ₁-responsive riboswitch.

The compound can have the structure of Formula II:

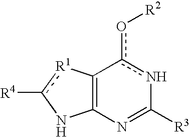

where R¹ can be CH, N, C—NH₂, C—CH₂—NH₂, C—CN, C—C(O)NH₂, C—CH═NH, C—CH₂—N(CH₃)₂, C-hydrogen bond donor, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl,

where R² is not present,

where R³ is NH₂, and

where R⁴ is not present.

The compound can have the structure of Formula II:

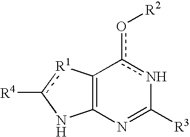

where R¹ is CH, N, C—NH₂, C—CH₂—NH₂, C—CN, C—C(O)NH₂, C—CH═NH, C—CH₂—N(CH₃)₂, or C-hydrogen bond donor,

where R² is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present,

where R³ is NH₂, and

where R⁴ is not present.

The compound can have the structure of Formula II:

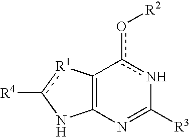

where R¹ is CH, N, C—NH₂, C—CH₂—NH₂, C—CN, C—C(O)NH₂, C—CH═NH, C—CH₂—N(CH₃)₂, or C-hydrogen bond donor,

where R² is not present,

where R³ is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present, and

where R⁴ is not present.

The compound can have the structure of Formula II:

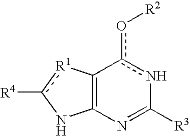

where R¹ is CH, N, C—NH₂, C—CH₂—NH₂, C—CN, C—C(O)NH₂, C—CH═NH, C—CH₂—N(CH₃)₂, or C-hydrogen bond donor,

where R² is not present,

where R³ is NH₂, and

where R⁴ is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present.

The compound can have the structure of Formula II:

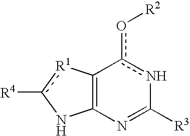

where R¹ is CH, N, C—NH₂, C—CH₂—NH₂, C—CN, C—C(O)NH₂, C—CH═NH, C—CH₂—N(CH₃)₂, or C-hydrogen bond donor,

where R² is where R² is not present,

where R³ is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present, and

where R⁴ is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present.

The compound can have the structure of Formula II:

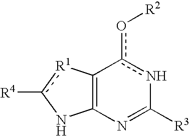

where R¹ is CH, N, C—NH₂, C—CH₂—NH₂, C—CN, C—C(O)NH₂, C—CH═NH, C—CH₂—N(CH₃)₂, or C-hydrogen bond donor,

where R² is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present,

where R³ is NH₂, and

where R⁴ is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present.

The compound can have the structure of Formula II:

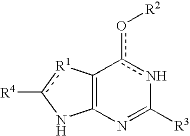

where R′ is CH, N, C—NH₂, C—CH₂—NH₂, C—CN, C—C(O)NH₂, C—CH═NH, C—CH₂—N(CH₃)₂, or C-hydrogen bond donor,

where R² is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present,

where R³ is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present, and

where R⁴ is not present.

The compound can have the structure of Formula II:

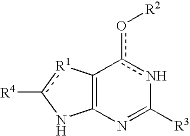

where R¹ is CH, N, C—NH₂, C—CH₂—NH₂, C—CN, C—C(O)NH₂, C—CH═NH, C—CH₂—N(CH₃)₂, or C-hydrogen bond donor,

where R² is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present,

where R³ is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present, and

where R⁴ is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present.

The cell can be identified as being in need of inhibited gene expression. The cell can be a bacterial cell. The compound can kill or inhibit the growth of the bacterial cell. The compound and the cell can be brought into contact by administering the compound to a subject. The cell can be a bacterial cell in the subject, wherein the compound kills or inhibits the growth of the bacterial cell. The subject can have a bacterial infection. The cell can contain a preQ₁-responsive riboswitch. The compound can be administered in combination with another antimicrobial compound. The compound can inhibit bacterial growth in a biofilm.

Also disclosed is a method of producing preQ₁, the method comprising: cultivating a mutant bacterial cell capable of producing preQ₁, wherein the mutant bacterial cell comprises a mutation in the preQ₁ riboswitch, which mutation increases preQ₁ production by the mutant bacterial cell in comparison to a cell not having the mutation; and isolating preQ₁ from the cell culture, thereby producing preQ₁. This method can yield at least a 10% increase in preQ₁ production compared to cultivating a bacterial cell that does not comprise the mutation in the preQ₁ riboswitch. This method can yield at least a 10% increase in preQ₁ production compared to cultivating a bacterial cell that does not comprise the mutation in the preQ₁ riboswitch. This method can yield at least a 25% increase in preQ₁ production compared to cultivating a bacterial cell that does not comprise the mutation in the preQ₁ riboswitch. The preQ₁ riboswitch can comprise a knockout mutation.

Further disclosed is a bacterial cell comprising a mutation in a preQ₁ riboswitch, which mutation measurably increases preQ₁ production by the cell when compared to a cell that does not have the mutation.

Also disclosed is a method of inhibiting bacterial cell growth, the method comprising: bringing into contact a cell and a compound that binds a preQ₁-responsive riboswitch, wherein the cell comprises a gene encoding an RNA comprising a preQ₁-responsive riboswitch, wherein the compound inhibits bacterial cell growth by binding to the preQ₁-responsive riboswitch, thereby limiting preQ₁ production. This method can yield at least a 10% decrease in bacterial cell growth compared to a cell that is not in contact with the compound. The compound and the cell can be brought into contact by administering the compound to a subject. The cell can be a bacterial cell in the subject, wherein the compound kills or inhibits the growth of the bacterial cell. The subject can have a bacterial infection. The compound can be administered in combination with another antimicrobial compound.

Disclosed is a method of detecting preQ₁ in a sample comprising: bringing a preQ₁-responsive riboswitch in contact with the sample; and detecting interaction between preQ₁ and the preQ₁-responsive riboswitch, wherein interaction between preQ₁ and the preQ₁-responsive riboswitch indicates the presence of preQ₁. The preQ₁-responsive riboswitch can be labeled.

Also disclosed is a method comprising inhibiting gene expression of a gene encoding an RNA comprising a riboswitch by bringing into contact a cell and a compound that was identified as a compound that inhibits gene expression of the gene by testing the compound for inhibition of gene expression of the gene, wherein the inhibition was via the riboswitch, wherein the riboswitch comprises a preQ₁-responsive riboswitch or a derivative of a preQ₁-responsive riboswitch.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows conserved sequences located in the 5′ UTRs of eubacterial genes involved in preQ₁ biosynthesis (SEQ ID NOS 5-42). Nucleotides that are >95% conserved (uppercase) and >80% conserved (lowercase) within the respective type are indicated in the consensus rows (R=A, G; Y═C, U). Regions marked by brackets in the structure row designate complementary sequences contributing to potential secondary structure elements P0 (lighter shading) and P1 (darker shading). Genes are indicated by locus tags from the original sequence files. Organism abbreviations: (Ban) Bacillus anthracis; (Bce) Bacillus cereus; (Bha) Bacillus halodurans; (Bsu) Bacillus subtilis; (Cac) Clostridium acetobutylicum; (Cpe) Clostridium perfringens; (Cte) Clostridium tetani; (Efa) Enterococcus faecalis; (Efm) Enterococcus faecium; (Exi) Exiguobacterium sp.; (Fnu) Fusobacterium nucleatum; (Gka) Geobacillus kaustophilus; (Hin) Haemophilus influenzae; (Lin) Listeria innocua; (Lme) Leuconostoc mesenteroides; (Lp1) Lactobacillus plantarum; (Nme1) Neisseria meningitidis MC58; (Nme2) Neisseria meningitidis Z2491; (Oih) Oceanobacillus iheyensis; (Pmu) Pasteurella multocida; (Sau) Staphylococcus aureus; (Sep) Staphylococcus epidermidis; (Sag) Streptococcus agalactiae; (Tte) Thermoanaerobacter tengcongensis; (Env) Environmental sequence IBEA_CTG_{—}2157609.

FIG. 2 shows queuosine biosynthesis in eubacteria. Enzymes known to participate in Q production are indicated, together with required cofactors (in parentheses), at the respective steps. Transformations for which specific corresponding enzymes remain to be identified are indicated by question marks.

FIG. 3 The 5′ UTR of the B. subtilis queCDEF mRNA undergoes structural modulation in response to preQ₁. (a) Primary and secondary structure consensus models corresponding to each type of the conserved domain associated with preQ₁ biosynthetic genes (SEQ ID NOS: 43, 44, and 45). Nucleotides in gray and black are more than 95% and 80% conserved, respectively, among the sequence representatives shown in FIG. 1. Less conserved regions, which may vary slightly in the number of nucleotides, are represented by circles or heavy lines. Locations of less conserved, putative stem elements are indicated in gray. R denotes A or G; Y denotes C or U. (b) Sequence and secondary structure model of 106 queC, a part of the B. subtilis queC 5′ UTR that contains predicted aptamer and terminator structures (SEQ ID NO: 45). Sites of spontaneous RNA cleavage that are responsive to or independent of the presence of preQ₁ are superimposed on the model and derived from structure probing analysis in c. There are 106 nucleotides between nucleotide 106 and the AUG start codon. Arrowhead indicates cleavage site corresponding to the shortest 5′³²P-labeled fragment visible in c. The 5′ terminal guanosyl residue is non-native and was introduced to facilitate transcription in vitro using T7 RNA polymerase. Constant scission was found for nucleotides 16-27, 31-38, 57, 58, 67, 77-81, 83 and 93-95 of SEQ ID NO:45. Reduced scission was found for nucleotides 50-53, 59, 60, 70, 71 and 74-76 of SEQ ID NO:45. Increased scission was found for nucleotides 55 and 56 of SEQ ID NO:45. (c) In-line probing analysis of 106 queC RNA reveals sites of increased and decreased strand scission (arrowheads) that are induced in the presence of preQ₁. Spontaneous RNA cleavage products from incubations in the absence (−) or presence of 1 μM or 10 μM preQ₁ were resolved by denaturing 10% PAGE. (NR) no reaction; (T1) partial digest with RNase T1; (⁻OH) partial alkaline digest; (Pre) precursor RNA. Selected bands in the T1 lane are indicated according to the positions of their 3′ terminal guanosyl residues.

FIG. 4 shows molecular discrimination by the preQ₁-binding RNA from the B. subtilis queC 5′ UTR. (a) Secondary structure model of the 52 queC RNA construct, which has been truncated relative to 106 queC to contain only phylogenetically conserved sequence and structural elements (SEQ ID NO: 46). The two 5′ terminal guanosine nucleotides are non-native residues that were introduced to facilitate transcription in vitro using T7 RNA polymerase. (b) Chemical structures of preQ₁ and preQ₀, and the respective apparent K_{d} values obtained with 52 queC. Shaded regions indicate chemical structure differences in comparison to preQ₁. Selected ring atoms are numbered on the preQ₁ structure. (c) Chemical structures of preQ₁-related compounds. The corresponding apparent K_{d} values were obtained using 52 queC, except in the cases of 2,6-diaminopurine and adenine, where the longer construct 80 queC was used (see FIG. 6a). Open circles denote apparent K_{d} values that are likely to be higher than indicated. (Concentrations in excess of 300 μM were not routinely tested in this analysis.) (d) The apparatus used in equilibrium dialysis experiments contained two chambers separated by a permeable membrane with a molecular weight cut-off (MWCO) of 5000 Daltons. The addition of ³H-guanine and 106 queC RNA individually to the respective chambers (top) results in a shift in the distribution of labeled guanine due to its retention in the RNA chamber (bottom right). In contrast, an equal distribution of ³H-guanine between the two chambers (bottom left) is expected if ligand-binding RNA is absent or if an unlabeled competitor ligand is present in excess. (e) Certain related purines, when added in molar excess, compete with ³H-guanine in binding 106 queC RNA. The extent of ³H-guanine sequestration in the RNA chamber is represented as the fraction of total added tritium that partitions to this compartment. Thus, a value of 0.5 is expected in cases where ³H-guanine is distributed equally between the two chambers, as occurs in the absence of 106 queC RNA or in the presence of excess unlabeled competitor. Conversely, a value approaching 1.0 is expected if retention of ³H-guanine occurs in the RNA chamber, as would result from equilibrium dialysis in the absence of unlabeled competitor or in the presence of unlabeled purines that do not function as competitors under the assay conditions (100 nM ³H-guanine, 20 μM RNA, 60 μM unlabeled analog). G, guanine; preQ₁, 7-aminomethyl-7-deazaguanine; A, adenine; 7 dG, 7-deazaguanine.

FIG. 5 shows determination of the minimal aptamer sequence required for binding of preQ₁. (a) Secondary structure model of the 36 queC construct, in which the P0 stem-loop has been deleted (SEQ ID NO: 47). Circled nucleotides indicate the 3′ termini of 36 queC and two derivative deletion constructs of progressively shorter lengths. Outlined residues indicate sites of ligand-induced structural modulation analyzed in subsequent panels. (b) In-line probing analysis reveals that conserved sequences near the 3′ end of 36 queC are required for preQ₁-induced structural modulation. Constructs were incubated in the absence (−) or presence (+) of 10 μM preQ₁. Other details and notations are as described in the legend to FIG. 3c. (c) Incubation of 36 queC RNA in the presence of increasing preQ₁ concentrations results in levels of spontaneous cleavage that decrease at site 1 and increase at site 2. (d) Graph showing the normalized fractions of cleaved RNA at sites 1 and 2 in c in relation to preQ₁ concentration.

FIG. 6 shows evidence for a Watson-Crick pairing interaction between preQ₁ and a conserved cytidyl residue of the aptamer. (a) Secondary structure model of 80 queC and two mutants, M1 and M2 (SEQ ID NO: 48). The two 5′ terminal guanosine nucleotides are non-native and were introduced to facilitate transcription in vitro. (b) In-line probing analyses of 80 queC RNA (WT), M1 and M2. Each construct was incubated in the absence of effector (−) and individually with 1 μM preQ₁ (Q₁), 200 μM guanine (G), 200 μM 2,6-diaminopurine (D), and 200 μM adenine (A). Other details and notations are as described in the legend to FIG. 3c. (c) Watson-Crick base pairing of preQ₁ with C34 as a putative determinant of 80 queC ligand selectivity. Proposed base-pairing interactions are shown between C34 of wild type 80 queC and preQ₁ (shaded background) or between U34 of the M1 construct and individual purine compounds tested in b.

FIG. 7 shows effects of variant preQ₁ riboswitches on genetic control in vivo. (a) Sequences corresponding to preQ₁ riboswitch constructs used in assays of reporter gene expression (SEQ ID NO: 49). (b) Regulation of β-galactosidase reporter gene expression by the wild-type riboswitch from B. subtilis queC and by mutant derivatives M3 through M9. Error bars are s.d. of three independent analyses.

FIG. 8 shows preQ₁ riboswitch locations and associated genes. GenBank record accession numbers and nucleotide positions are provided for each riboswitch element in the sequence alignment (FIG. 1). Predicted genes or operons downstream of each riboswitch element are designated by gene locus tags and accompanied by COG database assignments of general protein functions. The precise molecular functions corresponding to most genes in the preQ₁ regulon are currently unknown. Genes of the queCDEF operon have been implicated in Q biosynthesis, however (Reader 2004; Gaur 2005; Van Lanen 2005), and the specific chemical step catalyzed by QueF has been experimentally determined (Van Lanen 2005).

### DETAILED DESCRIPTION OF THE INVENTION

The disclosed methods and compositions can be understood more readily by reference to the following detailed description of particular embodiments and the Examples included therein and to the Figures and their previous and following description.

Messenger RNAs are typically thought of as passive carriers of genetic information that are acted upon by protein- or small RNA-regulatory factors and by ribosomes during the process of translation. It was discovered that certain mRNAs carry natural aptamer domains and that binding of specific metabolites directly to these RNA domains leads to modulation of gene expression. Natural riboswitches exhibit two surprising functions that are not typically associated with natural RNAs. First, the mRNA element can adopt distinct structural states wherein one structure serves as a precise binding pocket for its target metabolite. Second, the metabolite-induced allosteric interconversion between structural states causes a change in the level of gene expression by one of several distinct mechanisms. Riboswitches typically can be dissected into two separate domains: one that selectively binds the target (aptamer domain) and another that influences genetic control (expression platform). It is the dynamic interplay between these two domains that results in metabolite-dependent allosteric control of gene expression.

Distinct classes of riboswitches have been identified and are shown to selectively recognize activating compounds (referred to herein as trigger molecules). For example, coenzyme B₁₂, glycine, thiamine pyrophosphate (TPP), and flavin mononucleotide (FMN) activate riboswitches present in genes encoding key enzymes in metabolic or transport pathways of these compounds. The aptamer domain of each riboswitch class conforms to a highly conserved consensus sequence and structure. Thus, sequence homology searches can be used to identify related riboswitch domains. Riboswitch domains have been discovered in various organisms from bacteria, archaea, and eukarya.

### A. General Organization of Riboswitch RNAs

Bacterial riboswitch RNAs are genetic control elements that are located primarily within the 5′-untranslated region (5′-UTR) of the main coding region of a particular mRNA. Structural probing studies (discussed further below) reveal that riboswitch elements are generally composed of two domains: a natural aptamer (T. Hermann, D. J. Patel, Science 2000, 287, 820; L. Gold, et al., Annual Review of Biochemistry 1995, 64, 763) that serves as the ligand-binding domain, and an ‘expression platform’ that interfaces with RNA elements that are involved in gene expression (e.g. Shine-Dalgarno (SD) elements; transcription terminator stems). These conclusions are drawn from the observation that aptamer domains synthesized in vitro bind the appropriate ligand in the absence of the expression platform (see Examples 2, 3 and 6 of U.S. Application Publication No. 2005-0053951). Moreover, structural probing investigations suggest that the aptamer domain of most riboswitches adopts a particular secondary- and tertiary-structure fold when examined independently, that is essentially identical to the aptamer structure when examined in the context of the entire 5′ leader RNA. This indicates that, in many cases, the aptamer domain is a modular unit that folds independently of the expression platform (see Examples 2, 3 and 6 of U.S. Application Publication No. 2005-0053951).

Ultimately, the ligand-bound or unbound status of the aptamer domain is interpreted through the expression platform, which is responsible for exerting an influence upon gene expression. The view of a riboswitch as a modular element is further supported by the fact that aptamer domains are highly conserved amongst various organisms (and even between kingdoms as is observed for the TPP riboswitch), (N. Sudarsan, et al., RNA 2003, 9, 644) whereas the expression platform varies in sequence, structure, and in the mechanism by which expression of the appended open reading frame is controlled. For example, ligand binding to the TPP riboswitch of the tenA mRNA of B. subtilis causes transcription termination (A. S. Mironov, et al., Cell 2002, 111, 747). This expression platform is distinct in sequence and structure compared to the expression platform of the TPP riboswitch in the thiM mRNA from E. coli, wherein TPP binding causes inhibition of translation by a SD blocking mechanism (see Example 2 of U.S. Application Publication No. 2005-0053951). The TPP aptamer domain is easily recognizable and of near identical functional character between these two transcriptional units, but the genetic control mechanisms and the expression platforms that carry them out are very different.

Aptamer domains for riboswitch RNAs typically range from ˜70 to 170 nt in length (FIG. 11 of U.S. Application Publication No. 2005-0053951). This observation was somewhat unexpected given that in vitro evolution experiments identified a wide variety of small molecule-binding aptamers, which are considerably shorter in length and structural intricacy (T. Hermann, D. J. Patel, Science 2000, 287, 820; L. Gold, et al., Annual Review of Biochemistry 1995, 64, 763; M. Famulok, Current Opinion in Structural Biology 1999, 9, 324). Although the reasons for the substantial increase in complexity and information content of the natural aptamer sequences relative to artificial aptamers remains to be proven, this complexity is believed required to form RNA receptors that function with high affinity and selectivity. Apparent K_{D} values for the ligand-riboswitch complexes range from low nanomolar to low micromolar. It is also worth noting that some aptamer domains, when isolated from the appended expression platform, exhibit improved affinity for the target ligand over that of the intact riboswitch. (˜10 to 100-fold) (see Example 2 of U.S. Application Publication No. 2005-0053951). Presumably, there is an energetic cost in sampling the multiple distinct RNA conformations required by a fully intact riboswitch RNA, which is reflected by a loss in ligand affinity. Since the aptamer domain must serve as a molecular switch, this might also add to the functional demands on natural aptamers that might help rationalize their more sophisticated structures.

### B. The PreQ1 Riboswitch

A bioinformatics-based search for riboswitches yielded several candidate motifs in eubacteria. One of these motifs commonly resides in the 5′ untranslated regions of genes involved in the biosynthesis of queuosine (Q), a hypermodified nucleoside occupying the anticodon wobble position of certain tRNAs. It is herein shown that this structured RNA is part of a riboswitch selective for 7-aminomethyl-7-deazaguanine (preQ₁), an intermediate in Q biosynthesis. Compared to other natural metabolite-binding RNAs, the preQ₁ aptamer appears to have a simple structure, consisting of a single stem-loop and a short tail sequence that together are formed from as few as 34 nucleotides. Despite its small size, this aptamer is highly selective for its cognate ligand in vitro, and displays an affinity for preQ₁ in the low nanomolar range. Relatively compact RNA structures can therefore serve effectively as metabolite receptors to regulate gene expression.

Typically, the identity of a small molecule target of a particular riboswitch candidate has been inferred from the annotated functions of its associated genes. However, in instances where candidates are associated with genes of unknown function, the evaluation of prospective ligands is not straightforward. One candidate (ykvJ) that exemplifies this challenge was identified in a bioinformatics survey of noncoding regions from 91 microbial genomes (Barrick 2004). This element was discovered in several Firmicute species, and is associated most commonly with homologs of the B. subtilis ykvJKLM operon, whose protein products were uncharacterized. Furthermore, the conserved primary and secondary structure features of the ykvJ motif were confined to an unusually short span of nucleotides, whereas known riboswitches exhibit more extensive sequence conservation and more elaborate structures.

Subsequently, the gene families represented by the ykvJKLM operon were shown (Reader 2004) to be involved in the biosynthesis of Q, a hypermodified nucleoside found in eukarya and bacteria that occupies the anticodon wobble position of tRNAs specific for Asn, Asp, His and Tyr (Harada 1972). Certain genes in this operon (which have been renamed queC, -D, -E, and -F) are implicated specifically in the production of the Q precursor preQ₁ (Van Lanen 2005; Gaur 2005). In bacteria, preQ₁ is then transferred to the appropriate tRNAs by a tRNA-guanine transglycosylase (TGT) (Okada 1979), where it is further modified in situ to yield Q (Reuter 1991) or an aminoacylated derivative (Salazar 2004; Blaise 2004). Given the function of the queCDEF operon, preQ₁ or a related intermediate was considered as a potential ligand of the ykvJ (hereafter called queC) riboswitch candidate.

It is demonstrated herein (Example 1) that the 5′ UTR of the B. subtilis queCDEF operon selectively binds preQ₁ in vitro and controls expression of a reporter gene in vivo. Despite its unusually small size compared to other natural riboswitch aptamers, the ligand-binding domain of the queC riboswitch exhibits a dissociation constant (K_{d}) for preQ₁ in the nanomolar range. Also, by searching microbial genome databases, numerous additional examples of the queC motif were found, which define a widespread regulon whose expression can be modulated in response to intracellular concentrations of this important modified nucleobase.

### C. Riboswitch Regulation of Transcription Termination in Bacteria

Bacteria primarily make use of two methods for termination of transcription. Certain genes incorporate a termination signal that is dependent upon the Rho protein, (J. P. Richardson, Biochimica et Biophysica Acta 2002, 1577, 251). while others make use of Rho-independent terminators (intrinsic terminators) to destabilize the transcription elongation complex (I. Gusarov, E. Nudler, Molecular Cell 1999, 3, 495; E. Nudler, M. E. Gottesman, Genes to Cells 2002, 7, 755). The latter RNA elements are composed of a GC-rich stem-loop followed by a stretch of 6-9 uridyl residues. Intrinsic terminators are widespread throughout bacterial genomes (F. Lillo, et al., 2002, 18, 971), and are typically located at the 3′-termini of genes or operons. Interestingly, an increasing number of examples are being observed for intrinsic terminators located within 5′-UTRs.

Amongst the wide variety of genetic regulatory strategies employed by bacteria there is a growing class of examples wherein RNA polymerase responds to a termination signal within the 5′-UTR in a regulated fashion (T. M. Henkin, Current Opinion in Microbiology 2000, 3, 149). During certain conditions the RNA polymerase complex is directed by external signals either to perceive or to ignore the termination signal. Although transcription initiation might occur without regulation, control over mRNA synthesis (and of gene expression) is ultimately dictated by regulation of the intrinsic terminator. Presumably, one of at least two mutually exclusive mRNA conformations results in the formation or disruption of the RNA structure that signals transcription termination. A trans-acting factor, which in some instances is a RNA (F. J. Grundy, et al., Proceedings of the National Academy of Sciences of the United States of America 2002, 99, 11121; T. M. Henkin, C. Yanofsky, Bioessays 2002, 24, 700) and in others is a protein (J. Stulke, Archives of Microbiology 2002, 177, 433), is generally required for receiving a particular intracellular signal and subsequently stabilizing one of the RNA conformations. Riboswitches offer a direct link between RNA structure modulation and the metabolite signals that are interpreted by the genetic control machinery.

Most clinical antibacterial compounds target one of only four cellular processes (Wolfson 2006). Since bacteria have well developed resistance mechanisms to protect these processes (D′Costa 2006), it is useful to discover new targets that are vulnerable to drug intervention. One type of vulnerable process is the regulation of gene expression by riboswitches (Winkler 2005). Typically found in the 5′-UTRs of certain bacterial mRNAs, members of each known riboswitch class form a structured receptor (or “aptamer”) (Mandal 2004) that has evolved to bind a specific fundamental metabolite. In most cases, ligand binding regulates the expression of a gene or group of genes involved in the synthesis or transport of the bound metabolite. Because the biochemical pathways regulated by riboswitches are often essential for bacterial survival, repression of these pathways through riboswitch targeting can be lethal.

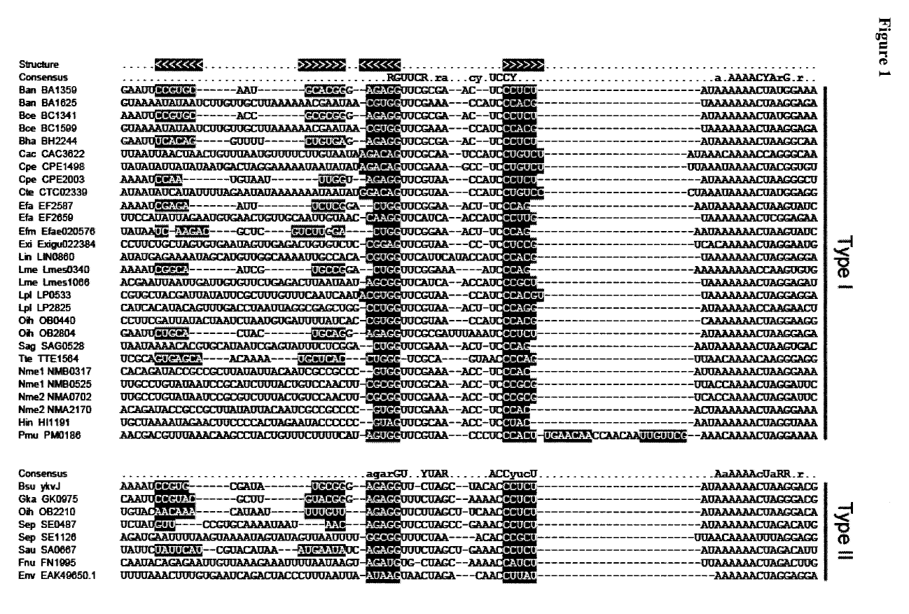

Several antibacterial metabolite analogs function by targeting riboswitches (Sudarsan 2003; Sudarsan 2005; Woolley 1943). For example, the antibacterial thiamine analog pyrithiamine (Woolley 1943) most likely functions by targeting a thiamine pyrophosphate-binding riboswitch (Sudarsan 2005). Similarly, the antibacterial lysine analog L-aminoethylcysteine (Shiota 1958) (AEC, FIG. 1b) binds to the lysC riboswitch from B. subtilis and represses the expression of a lysC-regulated reporter gene (Sudarsan 2006). Moreover, the lysC riboswitch is mutated in B. subtilis (Lu 1991) and Escherichia coli (Patte 1998) strains resistant to AEC.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference to each of various individual and collective combinations and permutation of these compounds can not be explicitly disclosed, each is specifically contemplated and described herein. For example, if a riboswitch or aptamer domain is disclosed and discussed and a number of modifications that can be made to a number of molecules including the riboswitch or aptamer domain are discussed, each and every combination and permutation of riboswitch or aptamer domain and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Riboswitches

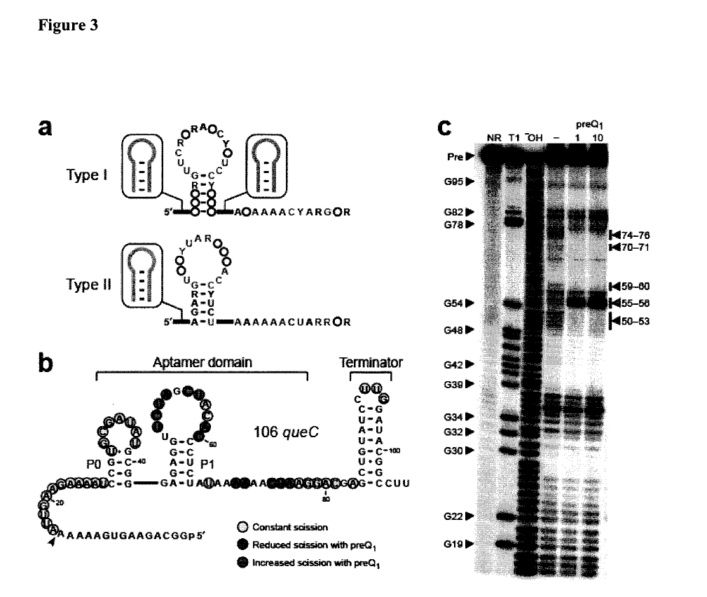

Riboswitches are expression control elements that are part of an RNA molecule to be expressed and that change state when bound by a trigger molecule. Riboswitches typically can be dissected into two separate domains: one that selectively binds the target (aptamer domain) and another that influences genetic control (expression platform domain). It is the dynamic interplay between these two domains that results in metabolite-dependent allosteric control of gene expression. Disclosed are isolated and recombinant riboswitches, recombinant constructs containing such riboswitches, heterologous sequences operably linked to such riboswitches, and cells and transgenic organisms harboring such riboswitches, riboswitch recombinant constructs, and riboswitches operably linked to heterologous sequences. The heterologous sequences can be, for example, sequences encoding proteins or peptides of interest, including reporter proteins or peptides. Preferred riboswitches are, or are derived from, naturally occurring riboswitches, such as naturally occurring preQ₁ riboswitches. The riboswitch can include or, optionlly, exclude, artificial aptamers. For example, artificial apatmers include apatamers that are designed or selected via in vitro evolution and/or in vitro selection. The riboswtiches can comprise the consensus sequence of naturally occurring riboswitches, such a consensus sequence of preQ₁ riboswitches. Consensus sequences of preQ₁ riboswitches are shown in FIG. 1 and FIG. 3a.

Disclosed is a riboswitch, wherein the riboswitch is a non-natural derivative of a naturally-occurring preQ₁-responsive riboswitch. The disclosed riboswitches, including the derivatives and recombinant forms thereof, generally can be from any source, including naturally occurring riboswitches and riboswitches designed de novo. Any such riboswitches can be used in or with the disclosed methods. However, different types of riboswitches can be defined and some such sub-types can be useful in or with particular methods (generally as described elsewhere herein). Types of riboswitches include, for example, naturally occurring riboswitches, derivatives and modified forms of naturally occurring riboswitches, chimeric riboswitches, and recombinant riboswitches. A naturally occurring riboswitch is a riboswitch having the sequence of a riboswitch as found in nature. Such a naturally occurring riboswitch can be an isolated or recombinant form of the naturally occurring riboswitch as it occurs in nature. That is, the riboswitch has the same primary structure but has been isolated or engineered in a new genetic or nucleic acid context. Chimeric riboswitches can be made up of, for example, part of a riboswitch of any or of a particular class or type of riboswitch and part of a different riboswitch of the same or of any different class or type of riboswitch; part of a riboswitch of any or of a particular class or type of riboswitch and any non-riboswitch sequence or component. Recombinant riboswitches are riboswitches that have been isolated or engineered in a new genetic or nucleic acid context.

Riboswitches can have single or multiple aptamer domains. Aptamer domains in riboswitches having multiple aptamer domains can exhibit cooperative binding of trigger molecules or can not exhibit cooperative binding of trigger molecules (that is, the aptamers need not exhibit cooperative binding). In the latter case, the aptamer domains can be said to be independent binders. Riboswitches having multiple aptamers can have one or multiple expression platform domains. For example, a riboswitch having two aptamer domains that exhibit cooperative binding of their trigger molecules can be linked to a single expression platform domain that is regulated by both aptamer domains. Riboswitches having multiple aptamers can have one or more of the aptamers joined via a linker. Where such aptamers exhibit cooperative binding of trigger molecules, the linker can be a cooperative linker.

Aptamer domains can be said to exhibit cooperative binding if they have a Hill coefficient n between x and x-1, where x is the number of aptamer domains (or the number of binding sites on the aptamer domains) that are being analyzed for cooperative binding. Thus, for example, a riboswitch having two aptamer domains (such as glycine-responsive riboswitches) can be said to exhibit cooperative binding if the riboswitch has Hill coefficient between 2 and 1. It should be understood that the value of x used depends on the number of aptamer domains being analyzed for cooperative binding, not necessarily the number of aptamer domains present in the riboswitch. This makes sense because a riboswitch can have multiple aptamer domains where only some exhibit cooperative binding.

Disclosed are chimeric riboswitches containing heterologous aptamer domains and expression platform domains. That is, chimeric riboswitches are made up an aptamer domain from one source and an expression platform domain from another source. The heterologous sources can be from, for example, different specific riboswitches, different types of riboswitches, or different classes of riboswitches. The heterologous aptamers can also come from non-riboswitch aptamers. The heterologous expression platform domains can also come from non-riboswitch sources.

Modified or derivative riboswitches can be produced using in vitro selection and evolution techniques. In general, in vitro evolution techniques as applied to riboswitches involve producing a set of variant riboswitches where part(s) of the riboswitch sequence is varied while other parts of the riboswitch are held constant. Activation, deactivation or blocking (or other functional or structural criteria) of the set of variant riboswitches can then be assessed and those variant riboswitches meeting the criteria of interest are selected for use or further rounds of evolution. Useful base riboswitches for generation of variants are the specific and consensus riboswitches disclosed herein. Consensus riboswitches can be used to inform which part(s) of a riboswitch to vary for in vitro selection and evolution.

Also disclosed are modified riboswitches with altered regulation. The regulation of a riboswitch can be altered by operably linking an aptamer domain to the expression platform domain of the riboswitch (which is a chimeric riboswitch). The aptamer domain can then mediate regulation of the riboswitch through the action of, for example, a trigger molecule for the aptamer domain. Aptamer domains can be operably linked to expression platform domains of riboswitches in any suitable manner, including, for example, by replacing the normal or natural aptamer domain of the riboswitch with the new aptamer domain. Generally, any compound or condition that can activate, deactivate or block the riboswitch from which the aptamer domain is derived can be used to activate, deactivate or block the chimeric riboswitch.

Also disclosed are inactivated riboswitches. Riboswitches can be inactivated by covalently altering the riboswitch (by, for example, crosslinking parts of the riboswitch or coupling a compound to the riboswitch). Inactivation of a riboswitch in this manner can result from, for example, an alteration that prevents the trigger molecule for the riboswitch from binding, that prevents the change in state of the riboswitch upon binding of the trigger molecule, or that prevents the expression platform domain of the riboswitch from affecting expression upon binding of the trigger molecule.

Also disclosed are biosensor riboswitches. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, biosensor riboswitches operably linked to a reporter RNA that encodes a protein that serves as or is involved in producing a signal can be used in vivo by engineering a cell or organism to harbor a nucleic acid construct encoding the riboswitch/reporter RNA. An example of a biosensor riboswitch for use in vitro is a riboswitch that includes a conformation dependent label, the signal from which changes depending on the activation state of the riboswitch. Such a biosensor riboswitch preferably uses an aptamer domain from or derived from a naturally occurring riboswitch. Biosensor riboswitches can be used in various situations and platforms. For example, biosensor riboswitches can be used with solid supports, such as plates, chips, strips and wells.

Also disclosed are modified or derivative riboswitches that recognize new trigger molecules. New riboswitches and/or new aptamers that recognize new trigger molecules can be selected for, designed or derived from known riboswitches. This can be accomplished by, for example, producing a set of aptamer variants in a riboswitch, assessing the activation of the variant riboswitches in the presence of a compound of interest, selecting variant riboswitches that were activated (or, for example, the riboswitches that were the most highly or the most selectively activated), and repeating these steps until a variant riboswitch of a desired activity, specificity, combination of activity and specificity, or other combination of properties results.

In general, any aptamer domain can be adapted for use with any expression platform domain by designing or adapting a regulated strand in the expression platform domain to be complementary to the control strand of the aptamer domain. Alternatively, the sequence of the aptamer and control strands of an aptamer domain can be adapted so that the control strand is complementary to a functionally significant sequence in an expression platform. For example, the control strand can be adapted to be complementary to the Shine-Dalgarno sequence of an RNA such that, upon formation of a stem structure between the control strand and the SD sequence, the SD sequence becomes inaccessible to ribosomes, thus reducing or preventing translation initiation. Note that the aptamer strand would have corresponding changes in sequence to allow formation of a P1 stem in the aptamer domain. In the case of riboswitches having multiple aptamers exhibiting cooperative binding, one the P1 stem of the activating aptamer (the aptamer that interacts with the expression platform domain) need be designed to form a stem structure with the SD sequence.

As another example, a transcription terminator can be added to an RNA molecule (most conveniently in an untranslated region of the RNA) where part of the sequence of the transcription terminator is complementary to the control strand of an aptamer domain (the sequence will be the regulated strand). This will allow the control sequence of the aptamer domain to form alternative stem structures with the aptamer strand and the regulated strand, thus either forming or disrupting a transcription terminator stem upon activation or deactivation of the riboswitch. Any other expression element can be brought under the control of a riboswitch by similar design of alternative stem structures.

For transcription terminators controlled by riboswitches, the speed of transcription and spacing of the riboswitch and expression platform elements can be important for proper control. Transcription speed can be adjusted by, for example, including polymerase pausing elements (e.g., a series of uridine residues) to pause transcription and allow the riboswitch to form and sense trigger molecules.

Disclosed are regulatable gene expression constructs comprising a nucleic acid molecule encoding an RNA comprising a preQ₁-responsive riboswitch operably linked to a coding region, wherein the riboswitch regulates expression of the RNA, wherein the riboswitch and coding region are heterologous. The riboswitch can comprise an aptamer domain and an expression platform domain, wherein the aptamer domain and the expression platform domain are heterologous. The riboswitch can comprise two or more aptamer domains and an expression platform domain, wherein at least one of the aptamer domains and the expression platform domain are heterologous. At least two of the aptamer domains can exhibit cooperative binding.

Disclosed are RNA molecules comprising heterologous riboswitch and coding region. That is, such RNA molecules are made up of a riboswitch from one source and a coding region from another source. The heterologous sources can be from, for example, different RNA molecules, different transcripts, RNA or transcripts from different genes, RNA or transcripts from different cells, RNA or transcripts from different organisms, RNA or transcripts from different species, natural sequences and artificial or engineered sequences, specific riboswitches, different types of riboswitches, or different classes of riboswitches.

As disclosed herein, the term “coding region” refers to any region of a nucleic acid that codes for amino acids. This can include both a nucleic acid strand that contains the codons or the template for codons and the complement of such a nucleic acid strand in the case of double stranded nuclec acid molecules. Regions of nucleic acids that are not coding regions can be referred to as noncoding regions. Messenger RNA molecules as transcribed typically include noncoding regions at both the 5′ and 3′ ends. Eucaryotic mRNA molecules can also include internal noncoding regions such as introns. Some types of RNA molecules do not include functional coding regions, such as tRNA and rRNA molecules. The expression of RNA molecules that do not include functional coding regions, which can be referred to as noncoding RNA molecules, can also be regulated or affected by the disclosed riboswitches. Thus, the disclosed riboswitches can be operably linked to a noncoding RNA molecule in any manner as disclosed herein for operable linkage of a riboswitch to a coding region. The riboswitch can regulate expression of such RNA as disclosed herein for regulation of RNA comprising a riboswitch operably linked to a coding region. The function of any nucleic acid molecule can also regulated or affected by the disclosed riboswitches. Examples include, but are not limited to, RNA, DNA, and artificial nucleic acids, including peptide nucleic acid (PNA), morpholino and locked nucleic acid (LNA), as well as glycol nucleic acid (GNA) and threose nucleic acid (TNA). In the disclosed method, the riboswitch can regulate expression of the coding region, expression of the encoded protein, expression of the noncoding RNA molecule, transcription of the RNA or of the coding regin, or translation of the encoded protein, for example.

1. Aptamer Domains

Aptamers are nucleic acid segments and structures that can bind selectively to particular compounds and classes of compounds. Riboswitches have aptamer domains that, upon binding of a trigger molecule result in a change in the state or structure of the riboswitch. In functional riboswitches, the state or structure of the expression platform domain linked to the aptamer domain changes when the trigger molecule binds to the aptamer domain. Aptamer domains of riboswitches can be derived from any source, including, for example, natural aptamer domains of riboswitches, artificial aptamers, engineered, selected, evolved or derived aptamers or aptamer domains. Aptamers in riboswitches generally have at least one portion that can interact, such as by forming a stem structure, with a portion of the linked expression platform domain. This stem structure will either form or be disrupted upon binding of the trigger molecule.

Consensus aptamer domains of a variety of natural riboswitches are shown in FIG. 11 of U.S. Application Publication No. 2005-0053951 and elsewhere herein. The consensus sequence and structure for the preQ₁ riboswitch can be found in FIG. 3. These aptamer domains (including all of the direct variants embodied therein) can be used in riboswitches. The consensus sequences and structures indicate variations in sequence and structure. Aptamer domains that are within the indicated variations are referred to herein as direct variants. These aptamer domains can be modified to produce modified or variant aptamer domains. Conservative modifications include any change in base paired nucleotides such that the nucleotides in the pair remain complementary. Moderate modifications include changes in the length of stems or of loops (for which a length or length range is indicated) of less than or equal to 20% of the length range indicated. Loop and stem lengths are considered to be “indicated” where the consensus structure shows a stem or loop of a particular length or where a range of lengths is listed or depicted. Moderate modifications include changes in the length of stems or of loops (for which a length or length range is not indicated) of less than or equal to 40% of the length range indicated. Moderate modifications also include and functional variants of unspecified portions of the aptamer domain.

Aptamer domains of the disclosed riboswitches can also be used for any other purpose, and in any other context, as aptamers. For example, aptamers can be used to control ribozymes, other molecular switches, and any RNA molecule where a change in structure can affect function of the RNA.

2. Expression Platform Domains

Expression platform domains are a part of riboswitches that affect expression of the RNA molecule that contains the riboswitch. Expression platform domains generally have at least one portion that can interact, such as by forming a stem structure, with a portion of the linked aptamer domain. This stem structure will either form or be disrupted upon binding of the trigger molecule. The stem structure generally either is, or prevents formation of, an expression regulatory structure. An expression regulatory structure is a structure that allows, prevents, enhances or inhibits expression of an RNA molecule containing the structure. Examples include Shine-Dalgarno sequences, initiation codons, transcription terminators, stability signals, and processing signals, such as RNA splicing junctions and control elements.

### B. Trigger Molecules

Trigger molecules are molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques).

### C. Compounds

Also disclosed are compounds, and compositions containing such compounds, that can activate, deactivate or block a riboswitch. Riboswitches function to control gene expression through the binding or removal of a trigger molecule. Compounds can be used to activate, deactivate or block a riboswitch. The trigger molecule for a riboswitch (as well as other activating compounds) can be used to activate a riboswitch. Compounds other than the trigger molecule generally can be used to deactivate or block a riboswitch. Riboswitches can also be deactivated by, for example, removing trigger molecules from the presence of the riboswitch. A riboswitch can be blocked by, for example, binding of an analog of the trigger molecule that does not activate the riboswitch.

Also disclosed are compounds for altering expression of an RNA molecule, or of a gene encoding an RNA molecule, where the RNA molecule includes a riboswitch. This can be accomplished by bringing a compound into contact with the RNA molecule. Riboswitches function to control gene expression through the binding or removal of a trigger molecule. Thus, subjecting an RNA molecule of interest that includes a riboswitch to conditions that activate, deactivate or block the riboswitch can be used to alter expression of the RNA. Expression can be altered as a result of, for example, termination of transcription or blocking of ribosome binding to the RNA. Binding of a trigger molecule can, depending on the nature of the riboswitch, reduce or prevent expression of the RNA molecule or promote or increase expression of the RNA molecule.

Also disclosed are compounds for regulating expression of an RNA molecule, or of a gene encoding an RNA molecule. Also disclosed are compounds for regulating expression of a naturally occurring gene or RNA that contains a riboswitch by activating, deactivating or blocking the riboswitch. If the gene is essential for survival of a cell or organism that harbors it, activating, deactivating or blocking the riboswitch can in death, stasis or debilitation of the cell or organism.

Also disclosed are compounds for regulating expression of an isolated, engineered or recombinant gene or RNA that contains a riboswitch by activating, deactivating or blocking the riboswitch. If the gene encodes a desired expression product, activating or deactivating the riboswitch can be used to induce expression of the gene and thus result in production of the expression product. If the gene encodes an inducer or repressor of gene expression or of another cellular process, activation, deactivation or blocking of the riboswitch can result in induction, repression, or de-repression of other, regulated genes or cellular processes. Many such secondary regulatory effects are known and can be adapted for use with riboswitches. An advantage of riboswitches as the primary control for such regulation is that riboswitch trigger molecules can be small, non-antigenic molecules.

Also disclosed are methods of identifying compounds that activate, deactivate or block a riboswitch. For example, compounds that activate a riboswitch can be identified by bringing into contact a test compound and a riboswitch and assessing activation of the riboswitch. If the riboswitch is activated, the test compound is identified as a compound that activates the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

Identification of compounds that block a riboswitch can be accomplished in any suitable manner. For example, an assay can be performed for assessing activation or deactivation of a riboswitch in the presence of a compound known to activate or deactivate the riboswitch and in the presence of a test compound. If activation or deactivation is not observed as would be observed in the absence of the test compound, then the test compound is identified as a compound that blocks activation or deactivation of the riboswitch.

Disclosed herein are analogs that interact with the preQ₁ riboswitch. Specifically, further modified versions of these compounds can have improved binding to the preQ₁ riboswitch by making new contacts to other functional groups in the RNA structure. Furthermore, modulation of bioavailability, toxicity, and synthetic ease (among other characteristics) can be tunable by making modifications in these two regions of the scaffold, as the structural model for the riboswitch shows many modifications are possible at these sites.

High-throughput screening can also be used to reveal entirely new chemical scaffolds that also bind to riboswitch RNAs either with standard or non-standard modes of molecular recognition. Since riboswitches are the first major form of natural metabolite-binding RNAs to be discovered, there has been little effort made previously to create binding assays that can be adapted for high-throughput screening. Multiple different approaches can be used to detect metabolite binding RNAs, including allosteric ribozyme assays using gel-based and chip-based detection methods, and in-line probing assays. Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound. This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For the purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol, as described below. The term “lower alkyl” is an alkyl group with 6 or fewer carbon atoms, e.g., methyl, ethyl, propyl, isopropyl, butyl, sec-butyl, iso-butyl, tert-butyl, pentyl, hexyl, and the like.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “halogenated alkyl” is used in another, it is not meant to imply that the term “alkyl” does not also refer to specific terms such as “halogenated alkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bonded through a single, terminal ether linkage; that is, an “alkoxy” group can be defined as —OA¹ where A² is alkyl as defined above.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula C(O)H. Throughout this specification “C(O)” is a short hand notation for C═O.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula

—C(O)OH. A “carboxylate” as used herein is represented by the formula —C(O)O⁻.

The term “ester” as used herein is represented by the formula —OC(O)A¹ or

—C(O)OA¹, where A¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxyl” as used herein is represented by the formula —OH.

The term “sulfo-oxo” as used herein is represented by the formulas —S(O)A¹ (i.e., “sulfonyl”), A′S(O)A² (i.e., “sulfoxide”), —S(O)₂A¹, A¹SO₂A² (i.e., “sulfone”),

—OS(O)₂A¹, or —OS(O)₂OA¹, where A₁ and A² can be hydrogen, an alkyl, halogenated alkyl, alkenyl, alkynyl, aryl, heteroaryl, cycloalkyl, cycloalkenyl, heterocycloalkyl, or heterocycloalkenyl group described above. Throughout this specification “S(O)” is a short hand notation for S═O.

The term “sulfonylamino” or “sulfonamide” as used herein is represented by the formula —S(O)₂NH—.

The term “thiol” as used herein is represented by the formula —SH.

As used herein, “Rⁿ” where n is some integer can independently possess one or more of the groups listed above. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Compounds useful with preQ₁-responsive riboswitches (and riboswitches derived from preQ₁-responsive riboswitches) include compounds represented by Formula I:

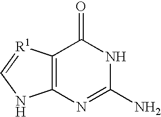

where R¹ is CH, N, C—NH₂, C—CH₂—NH₂, C—CN, C—C(O)NH₂, C—CH═NH, C—CH₂—N(CH₃)₂, or C-hydrogen bond donor,

wherein the cell comprises a gene encoding an RNA comprising a preQ₁-responsive riboswitch, wherein the compound inhibits expression of the gene by binding to the preQ₁-responsive riboswitch.

The compound can have the structure of Formula II:

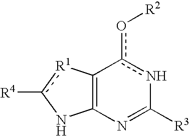

where R¹ can be CH, N, C—NH₂, C—CH₂—NH₂, C—CN, C—C(O)NH₂, C—CH═NH, C—CH₂—N(CH₃)₂, C-hydrogen bond donor, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl,

where R² is not present,

where R³ is NH₂, and

where R⁴ is not present.

The compound can have the structure of Formula II:

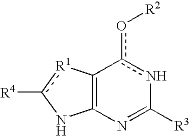

where R¹ is CH, N, C—NH₂, C—CH₂—NH₂, C—CN, C—C(O)NH₂, C—CH═NH, C—CH₂—N(CH₃)₂, or C-hydrogen bond donor,

where R² is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present,

where R³ is NH₂, and

where R⁴ is not present.

The compound can have the structure of Formula II:

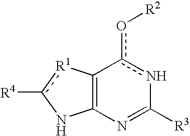

where R′ is CH, N, C—NH₂, C—CH₂—NH₂, C—CN, C—C(O)NH₂, C—CH═NH, C—CH₂—N(CH₃)₂, or C-hydrogen bond donor,

where R² is not present,

where R³ is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present, and

where R⁴ is not present.

The compound can have the structure of Formula II:

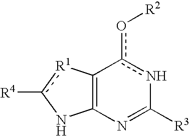

where R¹ is CH, N, C—NH₂, C—CH₂—NH₂, C—CN, C—C(O)NH₂, C—CH═NH, C—CH₂—N(CH₃)₂, or C-hydrogen bond donor,

where R² is not present,

where R³ is NH₂, and

where R⁴ is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present.

The compound can have the structure of Formula II:

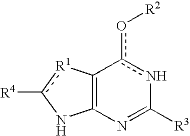

where R¹ is CH, N, C—NH₂, C—CH₂—NH₂, C—CN, C—C(O)NH₂, C—CH═NH, C—CH₂—N(CH₃)₂, or C-hydrogen bond donor,

where R² is where R² is not present,

where R³ is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present, and

where R⁴ is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present.

The compound can have the structure of Formula II:

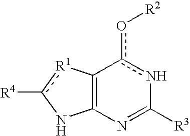

where R¹ is CH, N, C—NH₂, C—CH₂—NH₂, C—CN, C—C(O)NH₂, C—CH═NH, C—CH₂—N(CH₃)₂, or C-hydrogen bond donor,

where R² is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present,

where R³ is NH₂, and

where R⁴ is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present.

The compound can have the structure of Formula II:

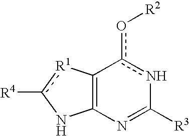

where R¹ is CH, N, C—NH₂, C—CH₂—NH₂, C—CN, C—C(O)NH₂, C—CH═NH, C—CH₂—N(CH₃)₂, or C-hydrogen bond donor,

where R² is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present,

where R³ is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present, and

where R⁴ is not present.

The compound can have the structure of Formula II:

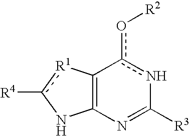

where R¹ is CH, N, C—NH₂, C—CH₂—NH₂, C—CN, C—C(O)NH₂, C—CH═NH, C—CH₂—N(CH₃)₂, or C-hydrogen bond donor,

where R² is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺₉ CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present,

where R³ is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present, and

where R⁴ is N, NH, NH₂⁺, NH₃⁺, O, OH, S, SH, C—R₅, CH—R₅, N—R₅, NH—R₅, O—R₅, or S—R₅, wherein R₅ is NH₂⁺, NH₃⁺, CO₂H, B(OH)₂, CH(NH₂)₂, C(NH₂)₂⁺, CNH₂NH₃⁺, C(NH₃₊)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present.

It is to be understood that while a particular moiety or group can be referred to herein as a hydrogen bond donor or acceptor, this terminology is used to merely categorize the various substituents for ease of reference. Such language should not be interpreted to mean that a particular moiety actually participates in hydrogen bonding with the riboswitch or some other compound. It is possible that, for example, a moiety referred to herein as a hydrogen bond acceptor (or donor) could solely or additionally be involved in hydrophobic, ionic, van de Waals, or other type of interaction with the riboswitch or other compound.

It is also understood that certain groups disclosed herein can be referred to herein as both a hydrogen bond acceptor and a hydrogen bond donor. For example, —OH can be a hydrogen bond donor by donating the hydrogen atom; —OH can also be a hydrogen bond acceptor through one or more of the nonbonded electron pairs on the oxygen atom. Thus, throughout the specification various moieties can be a hydrogen bond donor and acceptor and can be referred to as such.

Every compound within the above definition is intended to be and should be considered to be specifically disclosed herein. Further, every subgroup that can be identified within the above definition is intended to be and should be considered to be specifically disclosed herein. As a result, it is specifically contemplated that any compound, or subgroup of compounds can be either specifically included for or excluded from use or included in or excluded from a list of compounds. As an example, a group of compounds is contemplated where each compound is as defined above and is able to activate a preQ₁-responsive riboswitch.

It should be understood that particular contacts and interactions (such as hydrogen bond donation or acceptance) described herein for compounds interacting with riboswitches are preferred but are not essential for interaction of a compound with a riboswitch. For example, compounds can interact with riboswitches with less affinity and/or specificity than compounds having the disclosed contacts and interactions. Further, different or additional functional groups on the compounds can introduce new, different and/or compensating contacts with the riboswitches. For example, for preQ₁ riboswitches, large functional groups can be used. Such functional groups can have, and can be designed to have, contacts and interactions with other part of the riboswitch. Such contacts and interactions can compensate for contacts and interactions of the trigger molecules and core structure.

### D. Constructs, Vectors and Expression Systems

The disclosed preQ₁ riboswitches can be used with any suitable expression system. Recombinant expression is usefully accomplished using a vector, such as a plasmid. The vector can include a promoter operably linked to riboswitch-encoding sequence and RNA to be expression (e.g., RNA encoding a protein). The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes. A variety of prokaryotic and eukaryotic expression vectors suitable for carrying riboswitch-regulated constructs can be produced. Such expression vectors include, for example, pET, pET3d, pCR2.1, pBAD, pUC, and yeast vectors. The vectors can be used, for example, in a variety of in vivo and in vitro situation.

Viral vectors include adenovirus, adeno-associated virus, herpes virus, vaccinia virus, polio virus, AIDS virus, neuronal trophic virus, Sindbis and other RNA viruses, including these viruses with the HIV backbone. Also useful are any viral families which share the properties of these viruses which make them suitable for use as vectors. Retroviral vectors, which are described in Verma (1985), include Murine Maloney Leukemia virus, MMLV, and retroviruses that express the desirable properties of MMLV as a vector. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA.

A “promoter” is generally a sequence or sequences of DNA that function when in a relatively fixed location in regard to the transcription start site. A “promoter” contains core elements required for basic interaction of RNA polymerase and transcription factors and can contain upstream elements and response elements.

“Enhancer” generally refers to a sequence of DNA that functions at no fixed distance from the transcription start site and can be either 5′ (Laimins, 1981) or 3′ (Lusky et al., 1983) to the transcription unit. Furthermore, enhancers can be within an intron (Banerji et al., 1983) as well as within the coding sequence itself (Osborne et al., 1984). They are usually between 10 and 300 by in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

The vector can include nucleic acid sequence encoding a marker product. This marker product is used to determine if the gene has been delivered to the cell and once delivered is being expressed. Preferred marker genes are the E. coli lacZ gene which encodes β-galactosidase and green fluorescent protein.

In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern and Berg, 1982), mycophenolic acid, (Mulligan and Berg, 1980) or hygromycin (Sugden et al., 1985).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

1. Viral Vectors

Preferred viral vectors are Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Preferred retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors have higher transaction (ability to introduce genes) abilities than do most chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

i. Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

ii. Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A preferred viral vector is one based on an adenovirus which has had the E1 gene removed and these virons are generated in a cell line such as the human 293 cell line. In another preferred embodiment both the E1 and E3 genes are removed from the adenovirus genome.

Another type of viral vector is based on an adeno-associated virus (AAV). This defective parvovirus is a preferred vector because it can infect many cell types and is nonpathogenic to humans. AAV type vectors can transport about 4 to 5 kb and wild type

AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

2. Viral Promoters and Enhancers

Preferred promoters controlling transcription from vectors in mammalian host cells can be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 by in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

It is preferred that the promoter and/or enhancer region be active in all eukaryotic cell types. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In a preferred embodiment of the transcription unit, the polyadenylation region is derived from the SV40 early polyadenylation signal and consists of about 400 bases. It is also preferred that the transcribed units contain other standard sequences alone or in combination with the above sequences improve expression from, or stability of, the construct.

3. Markers

The vectors can include nucleic acid sequence encoding a marker product. This marker product is used to determine if the gene has been delivered to the cell and once delivered is being expressed. Preferred marker genes are the E. coli lacZ gene which encodes β-galactosidase and green fluorescent protein.

In some embodiments the marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR⁻ cells and mouse LTK⁻ cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### E. Biosensor Riboswitches

Also disclosed are biosensor riboswitches. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, preQ₁ biosensor riboswitches operably linked to a reporter RNA that encodes a protein that serves as or is involved in producing a signal can be used in vivo by engineering a cell or organism to harbor a nucleic acid construct encoding the preQ₁ riboswitch/reporter RNA. An example of a biosensor riboswitch for use in vitro is a riboswitch that includes a conformation dependent label, the signal from which changes depending on the activation state of the riboswitch. Such a biosensor riboswitch preferably uses an aptamer domain from or derived from a naturally occurring riboswitch, such as preQ₁.

### F. Reporter Proteins and Peptides

For assessing activation of a riboswitch, or for biosensor riboswitches, a reporter protein or peptide can be used. The reporter protein or peptide can be encoded by the RNA the expression of which is regulated by the riboswitch. The examples describe the use of some specific reporter proteins. The use of reporter proteins and peptides is well known and can be adapted easily for use with riboswitches. The reporter proteins can be any protein or peptide that can be detected or that produces a detectable signal. Preferably, the presence of the protein or peptide can be detected using standard techniques (e.g., radioimmunoassay, radio-labeling, immunoassay, assay for enzymatic activity, absorbance, fluorescence, luminescence, and Western blot). More preferably, the level of the reporter protein is easily quantifiable using standard techniques even at low levels. Useful reporter proteins include luciferases, green fluorescent proteins and their derivatives, such as firefly luciferase (FL) from Photinus pyralis, and Renilla luciferase (RL) from Renilla reniformis.

### G. Conformation Dependent Labels

Conformation dependent labels refer to all labels that produce a change in fluorescence intensity or wavelength based on a change in the form or conformation of the molecule or compound (such as a riboswitch) with which the label is associated. Examples of conformation dependent labels used in the context of probes and primers include molecular beacons, Amplifluors, FRET probes, cleavable FRET probes, TaqMan probes, scorpion primers, fluorescent triplex oligos including but not limited to triplex molecular beacons or triplex FRET probes, fluorescent water-soluble conjugated polymers, PNA probes and QPNA probes. Such labels, and, in particular, the principles of their function, can be adapted for use with riboswitches. Several types of conformation dependent labels are reviewed in Schweitzer and Kingsmore, Curr. Opin. Biotech. 12:21-27 (2001).

Stem quenched labels, a form of conformation dependent labels, are fluorescent labels positioned on a nucleic acid such that when a stem structure forms a quenching moiety is brought into proximity such that fluorescence from the label is quenched. When the stem is disrupted (such as when a riboswitch containing the label is activated), the quenching moiety is no longer in proximity to the fluorescent label and fluorescence increases. Examples of this effect can be found in molecular beacons, fluorescent triplex oligos, triplex molecular beacons, triplex FRET probes, and QPNA probes, the operational principles of which can be adapted for use with riboswitches.

Stem activated labels, a form of conformation dependent labels, are labels or pairs of labels where fluorescence is increased or altered by formation of a stem structure. Stem activated labels can include an acceptor fluorescent label and a donor moiety such that, when the acceptor and donor are in proximity (when the nucleic acid strands containing the labels form a stem structure), fluorescence resonance energy transfer from the donor to the acceptor causes the acceptor to fluoresce. Stem activated labels are typically pairs of labels positioned on nucleic acid molecules (such as riboswitches) such that the acceptor and donor are brought into proximity when a stem structure is formed in the nucleic acid molecule. If the donor moiety of a stem activated label is itself a fluorescent label, it can release energy as fluorescence (typically at a different wavelength than the fluorescence of the acceptor) when not in proximity to an acceptor (that is, when a stem structure is not formed). When the stem structure forms, the overall effect would then be a reduction of donor fluorescence and an increase in acceptor fluorescence. FRET probes are an example of the use of stem activated labels, the operational principles of which can be adapted for use with riboswitches.

### H. Detection Labels

To aid in detection and quantitation of riboswitch activation, deactivation or blocking, or expression of nucleic acids or protein produced upon activation, deactivation or blocking of riboswitches, detection labels can be incorporated into detection probes or detection molecules or directly incorporated into expressed nucleic acids or proteins. As used herein, a detection label is any molecule that can be associated with nucleic acid or protein, directly or indirectly, and which results in a measurable, detectable signal, either directly or indirectly. Many such labels are known to those of skill in the art. Examples of detection labels suitable for use in the disclosed method are radioactive isotopes, fluorescent molecules, phosphorescent molecules, enzymes, antibodies, and ligands.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as Quantum Dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH₃, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Useful fluorescent labels are fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

Labeled nucleotides are a useful form of detection label for direct incorporation into expressed nucleic acids during synthesis. Examples of detection labels that can be incorporated into nucleic acids include nucleotide analogs such as BrdUrd (5-bromodeoxyuridine, Hoy and Schimke, Mutation Research 290:217-230 (1993)), aminoallyldeoxyuridine (Henegariu et al., Nature Biotechnology 18:345-348 (2000)), 5-methylcytosine (Sano et al., Biochim. Biophys. Acta 951:157-165 (1988)), bromouridine (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (bromodeoxyuridine, BrdUrd, BrdU, BUdR, Sigma-Aldrich Co). Other useful nucleotide analogs for incorporation of detection label into DNA are AA-dUTP (aminoallyl-deoxyuridine triphosphate, Sigma-Aldrich Co.), and 5-methyl-dCTP (Roche Molecular Biochemicals). A useful nucleotide analog for incorporation of detection label into RNA is biotin-16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Detection labels that are incorporated into nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2-dioxetane-3-2′-(5′-chloro)tricyclo[3.3.1.1^{3,7}]decane]-4-yl)phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these detection labels are also considered detection labels. Any of the known detection labels can be used with the disclosed probes, tags, molecules and methods to label and detect activated or deactivated riboswitches or nucleic acid or protein produced in the disclosed methods. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. As used herein, detection molecules are molecules which interact with a compound or composition to be detected and to which one or more detection labels are coupled.

### I. Sequence Similarities

It is understood that as discussed herein the use of the terms homology and identity mean the same thing as similarity. Thus, for example, if the use of the word homology is used between two sequences (non-natural sequences, for example) it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed riboswitches, aptamers, expression platforms, genes and proteins herein, is through defining the variants and derivatives in terms of homology to specific known sequences. This identity of particular sequences disclosed herein is also discussed elsewhere herein. In general, variants of riboswitches, aptamers, expression platforms, genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to a stated sequence or a native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### J. Hybridization and Selective Hybridization

The term hybridization typically means a sequence driven interaction between at least two nucleic acid molecules, such as a primer or a probe and a riboswitch or a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting nucleic acid is in for example, 10 or 100 or 1000 fold excess. This type of assay can be performed at under conditions where both the limiting and non-limiting nucleic acids are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of nucleic acid that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions can provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### K. Nucleic Acids

There are a variety of molecules disclosed herein that are nucleic acid based, including, for example, riboswitches, aptamers, and nucleic acids that encode riboswitches and aptamers. The disclosed nucleic acids can be made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if a nucleic acid molecule is introduced into a cell or cell environment through for example exogenous delivery, it is advantageous that the nucleic acid molecule be made up of nucleotide analogs that reduce the degradation of the nucleic acid molecule in the cellular environment.

So long as their relevant function is maintained, riboswitches, aptamers, expression platforms and any other oligonucleotides and nucleic acids can be made up of or include modified nucleotides (nucleotide analogs). Many modified nucleotides are known and can be used in oligonucleotides and nucleic acids. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Base modifications often can be combined with for example a sugar modification, such as 2′-O-methoxyethyl, to achieve unique properties such as increased duplex stability. There are numerous U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference in its entirety, and specifically for their description of base modifications, their synthesis, their use, and their incorporation into oligonucleotides and nucleic acids.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C1 to C10, alkyl or C2 to C10 alkenyl and alkynyl. 2′ sugar modifications also include but are not limited to —O[(CH₂)n O]m CH₃, —O(CH₂)n OCH₃, —O(CH₂)n NH₂, —O(CH₂)n CH₃, —O(CH₂)n —ONH₂, and —O(CH₂)nON[(CH₂)n CH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications can also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S, Nucleotide sugar analogs can also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety, and specifically for their description of modified sugar structures, their synthesis, their use, and their incorporation into nucleotides, oligonucleotides and nucleic acids.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference its entirety, and specifically for their description of modified phosphates, their synthesis, their use, and their incorporation into nucleotides, oligonucleotides and nucleic acids.

It is understood that nucleotide analogs need only contain a single modification, but can also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to (base pair to) complementary nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference its entirety, and specifically for their description of phosphate replacements, their synthesis, their use, and their incorporation into nucleotides, oligonucleotides and nucleic acids.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science 254:1497-1500 (1991)).

Oligonucleotides and nucleic acids can be comprised of nucleotides and can be made up of different types of nucleotides or the same type of nucleotides. For example, one or more of the nucleotides in an oligonucleotide can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-β-methyl ribonucleotides. Such oligonucleotides and nucleic acids can be referred to as chimeric oligonucleotides and chimeric nucleic acids.

### L. Solid Supports

Solid supports are solid-state substrates or supports with which molecules (such as trigger molecules) and riboswitches (or other components used in, or produced by, the disclosed methods) can be associated. Riboswitches and other molecules can be associated with solid supports directly or indirectly. For example, analytes (e.g., trigger molecules, test compounds) can be bound to the surface of a solid support or associated with capture agents (e.g., compounds or molecules that bind an analyte) immobilized on solid supports. As another example, riboswitches can be bound to the surface of a solid support or associated with probes immobilized on solid supports. An array is a solid support to which multiple riboswitches, probes or other molecules have been associated in an array, grid, or other organized pattern.

Solid-state substrates for use in solid supports can include any solid material with which components can be associated, directly or indirectly. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

An array can include a plurality of riboswitches, trigger molecules, other molecules, compounds or probes immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification.

Although useful, it is not required that the solid support be a single unit or structure. A set of riboswitches, trigger molecules, other molecules, compounds and/or probes can be distributed over any number of solid supports. For example, at one extreme, each component can be immobilized in a separate reaction tube or container, or on separate beads or microparticles.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

Each of the components (for example, riboswitches, trigger molecules, or other molecules) immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

### M. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for detecting compounds, the kit comprising one or more biosensor riboswitches. The kits also can contain reagents and labels for detecting activation of the riboswitches.

### N. Mixtures

Disclosed are mixtures formed by performing or preparing to perform the disclosed method. For example, disclosed are mixtures comprising riboswitches and trigger molecules.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### O. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising biosensor riboswitches, a solid support and a signal-reading device.

### P. Data Structures and Computer Control

Disclosed are data structures used in, generated by, or generated from, the disclosed method. Data structures generally are any form of data, information, and/or objects collected, organized, stored, and/or embodied in a composition or medium. Riboswitch structures and activation measurements stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Methods

Disclosed are methods for activating, deactivating or blocking a riboswitch. Such methods can involve, for example, bringing into contact a riboswitch and a compound or trigger molecule that can activate, deactivate or block the riboswitch. Riboswitches function to control gene expression through the binding or removal of a trigger molecule. Compounds can be used to activate, deactivate or block a riboswitch. The trigger molecule for a riboswitch (as well as other activating compounds) can be used to activate a riboswitch. Compounds other than the trigger molecule generally can be used to deactivate or block a riboswitch. Riboswitches can also be deactivated by, for example, removing trigger molecules from the presence of the riboswitch. Thus, the disclosed method of deactivating a riboswitch can involve, for example, removing a trigger molecule (or other activating compound) from the presence or contact with the riboswitch. A riboswitch can be blocked by, for example, binding of an analog of the trigger molecule that does not activate the riboswitch.

Also disclosed are methods for altering expression of an RNA molecule, or of a gene encoding an RNA molecule, where the RNA molecule includes a riboswitch, by bringing a compound into contact with the RNA molecule. Riboswitches function to control gene expression through the binding or removal of a trigger molecule. Thus, subjecting an RNA molecule of interest that includes a riboswitch to conditions that activate, deactivate or block the riboswitch can be used to alter expression of the RNA. Expression can be altered as a result of, for example, termination of transcription or blocking of ribosome binding to the RNA. Binding of a trigger molecule can, depending on the nature of the riboswitch, reduce or prevent expression of the RNA molecule or promote or increase expression of the RNA molecule.

Also disclosed are methods for regulating expression of a naturally occurring gene or RNA that contains a riboswitch by activating, deactivating or blocking the riboswitch. If the gene is essential for survival of a cell or organism that harbors it, activating, deactivating or blocking the riboswitch can result in death, stasis or debilitation of the cell or organism. For example, activating a naturally occurring riboswitch in a naturally occurring gene that is essential to survival of a microorganism can result in death of the microorganism (if activation of the riboswitch turns off or represses expression). This is one basis for the use of the disclosed compounds and methods for antimicrobial and antibiotic effects. The compounds that have these antimicrobial effects are considered to be bacteriostatic or bacteriocidal.

Also disclosed are methods for selecting and identifying compounds that can activate, deactivate or block a riboswitch. Activation of a riboswitch refers to the change in state of the riboswitch upon binding of a trigger molecule. A riboswitch can be activated by compounds other than the trigger molecule and in ways other than binding of a trigger molecule. The term trigger molecule is used herein to refer to molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques). Non-natural trigger molecules can be referred to as non-natural trigger molecules.

Also disclosed are methods of killing or inhibiting bacteria, comprising contacting the bacteria with a compound disclosed herein or identified by the methods disclosed herein.

Also disclosed are methods of identifying compounds that activate, deactivate or block a riboswitch. For example, compounds that activate a riboswitch can be identified by bringing into contact a test compound and a riboswitch and assessing activation of the riboswitch. If the riboswitch is activated, the test compound is identified as a compound that activates the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

In addition to the methods disclosed elsewhere herein, identification of compounds that block a riboswitch can be accomplished in any suitable manner. For example, an assay can be performed for assessing activation or deactivation of a riboswitch in the presence of a compound known to activate or deactivate the riboswitch and in the presence of a test compound. If activation or deactivation is not observed as would be observed in the absence of the test compound, then the test compound is identified as a compound that blocks activation or deactivation of the riboswitch.

Also disclosed are methods of detecting compounds using biosensor riboswitches. The method can include bringing into contact a test sample and a biosensor riboswitch and assessing the activation of the biosensor riboswitch. Activation of the biosensor riboswitch indicates the presence of the trigger molecule for the biosensor riboswitch in the test sample. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, preQ₁ biosensor riboswitches operably linked to a reporter RNA that encodes a protein that serves as or is involved in producing a signal can be used in vivo by engineering a cell or organism to harbor a nucleic acid construct encoding the riboswitch/reporter RNA. An example of a biosensor riboswitch for use in vitro is a preQ₁ riboswitch that includes a conformation dependent label, the signal from which changes depending on the activation state of the riboswitch. Such a biosensor riboswitch preferably uses an aptamer domain from or derived from a naturally occurring preQ₁ riboswitch.

Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound. This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

Further disclosed is a method of detecting a compound of interest, the method comprising: bringing into contact a sample and a riboswitch, wherein the riboswitch is activated by the compound of interest, wherein the riboswitch produces a signal when activated by the compound of interest, wherein the riboswitch produces a signal when the sample contains the compound of interest, wherein the riboswitch comprises a preQ₁-responsive riboswitch or a derivative of a preQ₁-responsive riboswitch. The riboswitch can change conformation when activated by the compound of interest, wherein the change in conformation produces a signal via a conformation dependent label. The riboswitch can also change conformation when activated by the compound of interest, wherein the change in conformation causes a change in expression of an RNA linked to the riboswitch, wherein the change in expression produces a signal. The signal can be produced by a reporter protein expressed from the RNA linked to the riboswitch.

Specifically, disclosed is a method of detecting preQ₁ in a sample comprising: bringing a preQ₁-responsive riboswitch in contact with the sample; and detecting interaction between preQ₁ and the preQ₁-responsive riboswitch, wherein interaction between preQ₁ and the preQ₁-responsive riboswitch indicates the presence of preQ₁. The preQ₁-responsive riboswitch can be labeled.

Disclosed is a method comprising: (a) testing a compound for inhibition of gene expression of a gene encoding an RNA comprising a riboswitch, wherein the inhibition is via the riboswitch, wherein the riboswitch comprises a preQ₁-responsive riboswitch or a derivative of a preQ₁-responsive riboswitch, (b) inhibiting gene expression by bringing into contact a cell and a compound that inhibited gene expression in step (a), wherein the cell comprises a gene encoding an RNA comprising a riboswitch, wherein the compound inhibits expression of the gene by binding to the riboswitch.

Also disclosed is a method comprising inhibiting gene expression of a gene encoding an RNA comprising a riboswitch by bringing into contact a cell and a compound that was identified as a compound that inhibits gene expression of the gene by testing the compound for inhibition of gene expression of the gene, wherein the inhibition was via the riboswitch, wherein the riboswitch comprises a preQ₁-responsive riboswitch or a derivative of a preQ₁-responsive riboswitch.

### A. Identification of Antimicrobial Compounds

Riboswitches are a new class of structured RNAs that have evolved for the purpose of binding small organic molecules. The natural binding pocket of riboswitches can be targeted with metabolite analogs or by compounds that mimic the shape-space of the natural metabolite. The small molecule ligands of riboswitches provide useful sites for derivitization to produce drug candidates. Distribution of some riboswitches is shown in Table 1 of U.S. Application Publication No. 2005-0053951. Once a class of riboswitch has been identified and its potential as a drug target assessed, such as the preQ₁ riboswitch, candidate molecules can be identified.

The emergence of drug-resistant stains of bacteria highlights the need for the identification of new classes of antibiotics. Anti-riboswitch drugs represent a mode of anti-bacterial action that is of considerable interest for the following reasons. Riboswitches control the expression of genes that are critical for fundamental metabolic processes. Therefore manipulation of these gene control elements with drugs yields new antibiotics. These antimicrobial agents can be considered to be bacteriostatic, or bacteriocidal. Riboswitches also carry RNA structures that have evolved to selectively bind metabolites, and therefore these RNA receptors make good drug targets as do protein enzymes and receptors. Furthermore, it has been shown that two antimicrobial compounds (discussed above) kill bacteria by deactivating the antibiotics resistance to emerge through mutation of the RNA target.

A compound can be identified as activating a riboswitch or can be determined to have riboswitch activating activity if the signal in a riboswitch assay is increased in the presence of the compound by at least 1 fold, 2 fold, 3 fold, 4 fold, 5 fold, 50%, 75%, 100%, 125%, 150%, 175%, 200%, 250%, 300%, 400%, or 500% compared to the same riboswitch assay in the absence of the compound (that is, compared to a control assay). The riboswitch assay can be performed using any suitable riboswitch construct. Riboswitch constructs that are particularly useful for riboswitch activation assays are described elsewhere herein. The identification of a compound as activating a riboswitch or as having a riboswitch activation activity can be made in terms of one or more particular riboswitches, riboswitch constructs or classes of riboswitches. For convenience, compounds identified as activating a preQ₁ riboswitch or having riboswitch activating activity for a preQ₁ riboswitch can be so identified for particular preQ₁ riboswitches, such as the preQ₁ riboswitches found in Bacillus anthracis or B. subtilis.

### B. Methods of Using Antimicrobial Compounds

Disclosed herein are in vivo and in vitro anti-bacterial methods. By “anti-bacterial” is meant inhibiting or preventing bacterial growth, killing bacteria, or reducing the number of bacteria. Thus, disclosed is a method of inhibiting or preventing bacterial growth comprising contacting a bacterium with an effective amount of one or more compounds disclosed herein. Additional structures for the disclosed compounds are provided herein.

Disclosed is a method of inhibiting bacterial cell growth, the method comprising: bringing into contact a cell and a compound that binds a preQ₁-responsive riboswitch, wherein the cell comprises a gene encoding an RNA comprising a preQ₁-responsive riboswitch, wherein the compound inhibits bacterial cell growth by binding to the preQ₁-responsive riboswitch, thereby limiting preQ₁ production. This method can yield at least a 10% decrease in bacterial cell growth compared to a cell that is not in contact with the compound. The compound and the cell can be brought into contact by administering the compound to a subject. The cell can be a bacterial cell in the subject, wherein the compound kills or inhibits the growth of the bacterial cell. The subject can have a bacterial infection. The compound can be administered in combination with another antimicrobial compound.

The bacteria can be any bacteria, such as bacteria from the genus Bacillus, Acinetobacter, Actinobacillus, Clostridium, Desulfitobacterium, Enterococcus, Erwinia, Escherichia, Exiguobacterium, Fusobacterium, Geobacillus, Haemophilus, Klebsiella, Idiomarina, Lactobacillus, Lactococcus, Leuconostoc, Listeria, Moorella, Mycobacterium, Oceanobacillus, Oenococcus, Pasteurella, Pediococcus, Pseudomonas, Shewanella, Shigella, Solibacter, Staphylococcus, Streptococcus, Thermoanaerobacter, Thermotoga, and Vibrio, for example. The bacteria can be, for example, Actinobacillus pleuropneumoniae, Bacillus anthracis, Bacillus cereus, Bacillus clausii, Bacillus halodurans, Bacillus licheniformis, Bacillus subtilis, Bacillus thuringiensis, Clostridium acetobutylicum, Clostridium difficile, Clostridium perfringens, Clostridium tetani, Clostridium thermocellum, Desulfitobacterium hafniense, Enterococcus faecalis, Erwinia carotovora, Escherichia coli, Exiguobacterium sp., Fusobacterium nucleatum, Geobacillus kaustophilus, Haemophilus ducreyi, Haemophilus influenzae, Haemophilus somnus, Idiomarina loihiensis, Lactobacillus acidophilus, Lactobacillus casei, Lactobacillus delbrueckii, Lactobacillus gasseri, Lactobacillus johnsonii, Lactobacillus plantarum, Lactococcus lactis, Leuconostoc mesenteroides, Listeria innocua, Listeria monocytogenes, Moorella thermoacetica, Oceanobacillus iheyensis, Oenococcus oeni, Pasteurella multocida, Pediococcus pentosaceus, Shewanella oneidensis, Shigella flexneri, Solibacter usitatus, Staphylococcus aureus, Staphylococcus epidermidis, Thermoanaerobacter tengcongensis, Thermotoga maritima, Vibrio cholerae, Vibrio fischeri, Vibrio parahaemolyticus, or Vibrio vulnificus. Bacterial growth can also be inhibited in any context in which bacteria are found. For example, bacterial growth in fluids, biofilms, and on surfaces can be inhibited. The compounds disclosed herein can be administered or used in combination with any other compound or composition. For example, the disclosed compounds can be administered or used in combination with another antimicrobial compound.

“Inhibiting bacterial growth” is defined as reducing the ability of a single bacterium to divide into daughter cells, or reducing the ability of a population of bacteria to form daughter cells. The ability of the bacteria to reproduce can be reduced by about 10%, about 20%, about 30%, about 40%, about 50%, about 60%, about 70%, about 80%, about 90%, or 100% or more.

Also provided is a method of inhibiting the growth of and/or killing a bacterium or population of bacteria comprising contacting the bacterium with one or more of the compounds disclosed and described herein.

“Killing a bacterium” is defined as causing the death of a single bacterium, or reducing the number of a plurality of bacteria, such as those in a colony. When the bacteria are referred to in the plural form, the “killing of bacteria” is defined as cell death of a given population of bacteria at the rate of 10% of the population, 20% of the population, 30% of the population, 40% of the population, 50% of the population, 60% of the population, 70% of the population, 80% of the population, 90% of the population, or less than or equal to 100% of the population.

The compounds and compositions disclosed herein have anti-bacterial activity in vitro or in vivo, and can be used in conjunction with other compounds or compositions, which can be bactericidal as well.

By the term “therapeutically effective amount” of a compound as provided herein is meant a nontoxic but sufficient amount of the compound to provide the desired reduction in one or more symptoms. As will be pointed out below, the exact amount of the compound required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount may be determined by one of ordinary skill in the art using only routine experimentation.

The compositions and compounds disclosed herein can be administered in vivo in a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions or compounds disclosed herein can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition or compounds, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The compositions and compounds disclosed herein can be used therapeutically in combination with a pharmaceutically acceptable carrier. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Therapeutic compositions as disclosed herein may also be delivered by the use of monoclonal antibodies as individual carriers to which the compound molecules are coupled. The therapeutic compositions of the present disclosure may also be coupled with soluble polymers as targetable drug carriers. Such polymers can include, but are not limited to, polyvinyl-pyrrolidone, pyran copolymer, polyhydroxypropylmethacryl-amidephenol, polyhydroxyethylaspartamidephenol, or polyethyl-eneoxidepolylysine substituted with palmitoyl residues. Furthermore, the therapeutic compositions of the present disclosure may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

Preferably at least about 3%, more preferably about 10%, more preferably about 20%, more preferably about 30%, more preferably about 50%, more preferably 75% and even more preferably about 100% of the bacterial infection is reduced due to the administration of the compound. A reduction in the infection is determined by such parameters as reduced white blood cell count, reduced fever, reduced inflammation, reduced number of bacteria, or reduction in other indicators of bacterial infection. To increase the percentage of bacterial infection reduction, the dosage can increase to the most effective level that remains non-toxic to the subject.

As used throughout, “subject” refers to an individual. Preferably, the subject is a mammal such as a non-human mammal or a primate, and, more preferably, a human. “Subjects” can include domesticated animals (such as cats, dogs, etc.), livestock (e.g., cattle, horses, pigs, sheep, goats, etc.), laboratory animals (e.g., mouse, rabbit, rat, guinea pig, etc.) and fish.

A “bacterial infection” is defined as the presence of bacteria in a subject or sample. Such bacteria can be an outgrowth of naturally occurring bacteria in or on the subject or sample, or can be due to the invasion of a foreign organism.

The compounds disclosed herein can be used in the same manner as antibiotics. Uses of antibiotics are well established in the art. One example of their use includes treatment of animals. When needed, the disclosed compounds can be administered to the animal via injection or through feed or water, usually with the professional guidance of a veterinarian or nutritionist. They are delivered to animals either individually or in groups, depending on the circumstances such as disease severity and animal species. Treatment and care of the entire herd or flock may be necessary if all animals are of similar immune status and all are exposed to the same disease-causing microorganism.

Another example of a use for the compounds includes reducing a microbial infection of an aquatic animal, comprising the steps of selecting an aquatic animal having a microbial infection, providing an antimicrobial solution comprising a compound as disclosed, chelating agents such as EDTA, TRIENE, adding a pH buffering agent to the solution and adjusting the pH thereof to a value of between about 7.0 and about 9.0, immersing the aquatic animal in the solution and leaving the aquatic animal therein for a period that is effective to reduce the microbial burden of the animal, removing the aquatic animal from the solution and returning the animal to water not containing the solution. The immersion of the aquatic animal in the solution containing the EDTA, a compound as disclosed, and TRIENE and pH buffering agent may be repeated until the microbial burden of the animal is eliminated. (U.S. Pat. No. 6,518,252).

Other uses of the compounds disclosed herein include, but are not limited to, dental treatments and purification of water (this can include municipal water, sewage treatment systems, potable and non-potable water supplies, and hatcheries, for example).

### C. Methods of Producing PreQ₁

Disclosed herein is a method of producing preQ₁, the method comprising: cultivating a mutant bacterial cell capable of producing preQ₁, wherein the mutant bacterial cell comprises a mutation in the preQ₁ riboswitch, which mutation increases preQ₁ production by the mutant bacterial cell in comparison to a cell not having the mutation; and isolating preQ₁ from the cell culture, thereby producing preQ₁.

The mutant bacterial cell can be a knockout mutant, wherein the cell cannot produce the preQ₁ riboswitch. The cell can have the region coding for the preQ₁ riboswitch removed completely. Production of preQ₁ can be increased by 10, 20, 30, 40, 50, 60, 70, 80, 90% or more. This can be measured by the overall amount of preQ₁ compared to that produced by a cell that has an intact preQ₁ riboswitch.

Further disclosed is a bacterial cell comprising a mutation in a preQ₁ riboswitch, which mutation measurably increases preQ₁ production by the cell when compared to a cell that does not have the mutation. By “measurably increases” is meant a 10, 20, 30, 40, 50, 60, 70, 80, 90% or more increase in production of preQ₁.

### EXAMPLES

### Example 1

### A Riboswitch Selective for the Queuosine Precursor preQ₁ Contains an Unusually Small Aptamer Domain

i. RESULTS

a. Widespread Phylogenetic Conservation of the queC Motif

The initially reported examples of the queC motif were restricted to only a few bacterial species of the orders Bacillales and Clostridia. The majority of the sequence conservation among these examples occurred within a span of fewer than 40 nt. The apparent limited distribution of this motif and its relatively small size are distinct from known riboswitches, which tend to be phylogenetically more widespread and contain conserved sequence elements and structural features that require more nucleotides.

To determine whether the queC element might be more widely distributed, a search algorithm based on a revised secondary structure model was employed. The small number of queC RNA examples originally identified contained regions that were complementary but that were also absolutely conserved in sequence. Although extensive sequence conservation within stem structures is not unprecedented among riboswitches, sequence variability is more typical as long as stem integrity is preserved. Thus, despite their base-pairing potential, these absolutely conserved regions were not previously represented as a secondary structure element.

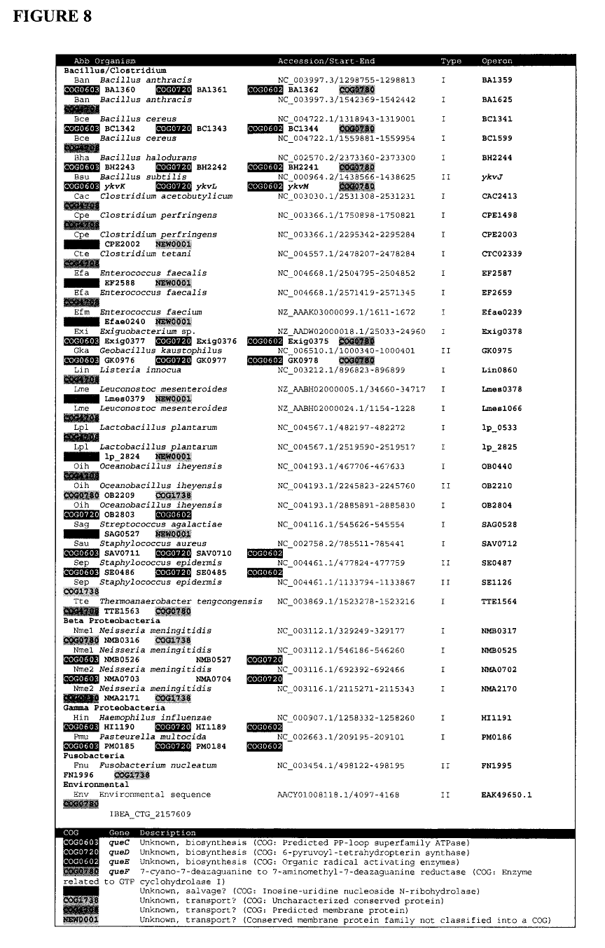

When the search constraints in these regions were relaxed, so that the principal requirement was no longer primary structure but secondary structure, a number of additional representatives of the queC motif were uncovered among Firmicutes, Proteobacteria and Fusobacteria (FIG. 1 and FIG. 8). Alignment of these sequences revealed two closely spaced stem-loop structures, only one of which is consistently present, and a highly conserved sequence residing immediately 3′ of the second stem-loop. Due to its sporadic occurrence, the 5′-most stem is referred to as P0, while the downstream, absolutely conserved helical element is termed P1. In cases where P0 is present, the corresponding loop sequences (L0) exhibit no sequence conservation. This observation, together with the apparent absence of P0 in many examples, suggests this putative structural element is not essential for the function of the queC motif. The numbers of intervening nucleotides between the P0 and P1 elements, and between P1 and the 3′ conserved segment, indicate that the distances between these elements are rigidly constrained, although in one instance (Pasteurella multocida; FIG. 1), insertion of an additional putative stem-loop is observed immediately 3′ to P1.

The majority of the conserved sequences reside within L1 and in the adenosine-rich 3′ tailing segment. Representatives of this motif can be segregated into two types based on differences in their L1 sequences, which range in length from 10 to 15 nt (FIG. 1). Although some features of the P1 substructure are conserved in all examples, the existence of two subtypes of the queC motif is evident from distinct signature sequences within the corresponding loop. Interestingly, the queC motif from Thermoanaerobacter tengcongensis carries an L1 sequence that exhibits features of both subtypes, suggesting that the different signature sequences can be blended to some extent.

b. The queC Motif is Associated with Q Biosynthetic Genes

Representatives comprising the expanded set of queC motifs were striking in their sequence and structural conservation, and because they were found to be consistently associated with homologous genes from a diverse collection of bacteria (FIG. 8). These attributes strongly implied a regulatory role for this RNA motif, although further analysis was hampered by the lack of characterization of the genes in the apparent regulon. This problem was resolved when it was reported that the gene families usually affiliated with the RNA (FIG. 8) are involved in Q biosynthesis (Reader 2004), and the search for a possible riboswitch ligand was focused on the metabolic products of this biosynthetic pathway.

Q and its derivatives are hypermodified versions of guanosine that are found widely among eukaryotes and eubacteria, where Q has been implicated in a broad range of physiological processes (Iwata-Reuyl 2003). Among eubacteria, it has been demonstrated that the Q modification is required for phenomena such as virulence in the pathogen Shigella flexneri (Durand 1994) and viability during stationary phase in Escherichia coli (Noguchi 1982; Frey 1989). The collection of disparate phenotypes resulting from Q deficiency is generally attributed to adverse effects on translational fidelity (Meier 1985; Bienz 1981; Urbonavicius 2001), an interpretation that is consistent with the occurrence of Q in the anticodon loop of some tRNAs.

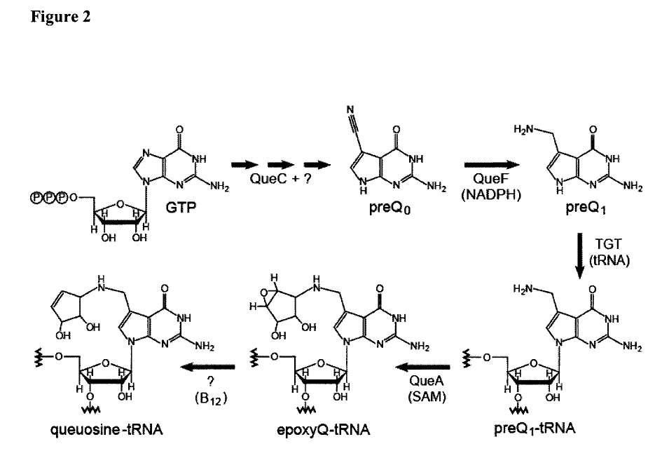

In eubacteria, de novo Q biosynthesis requires synthesis of the free nucleobase (FIG. 2), with guanosine triphosphate (GTP) serving as the starting material (Kuchino. The first known intermediate in the biosynthetic pathway is 7-cyano-7-deazaguanine (preQ₀) (Okada 1978), which subsequently is converted to preQ₁ in an NADPH-dependent reduction catalyzed by QueF (Van Lanen 1972). In a guanine-exchange reaction mediated by the TGT enzyme, preQ₁ is inserted at the appropriate position in the anticodons of the relevant tRNAs (Okada 1979; Reuter 1991). Following the incorporation of preQ₁ into tRNA, Q production continues in situ, first with the addition of an epoxycyclopentandiol ring derived from the ribosyl moiety of S-adenosylmethionine (SAM) (Slany 1994; Frey 1988), and then with an apparent coenzyme B₁₂-dependent step in which the epoxide is reduced to yield Q (Frey 1988). Interestingly, recent studies indicate that Q is subjected to yet further modification in some eubacteria by glutamylation of the cyclopentendiol moiety (Salazar 2004; Blaise 2004). The small molecule intermediates preQ₀ and preQ₁ were considered as candidate ligands for the RNA motif. In support of this possibility, recent studies have implicated the queC, -D, -E, and —F gene families, representatives of which are commonly associated with queC motifs, in biochemical steps upstream of preQ₁ (Reader 2004; Van Lanen 2005; Gaur 2005).

c. The queC Motif from B. subtilis Binds preQ₁

Representatives of the queC element can be sorted into one of two types based on distinct signature sequences within the conserved loop (FIGS. 1 and 3a). A 106 nt RNA containing the queC motif from B. subtilis (termed 106 queC; FIG. 3b) were examined, whose nucleotide sequence conforms to the type II consensus. This RNA construct included the conserved sequence and structural elements of the queC motif as well as a stem-loop with characteristics of an intrinsic transcription terminator (Gusarov 1999; Yarnell 1999) (FIG. 3b).

To test the possibility of a protein-independent interaction between the B. subtilis queC motif and preQ₁, 5′³²P-labeled 106 queC RNA was subjected to in-line probing analysis, which takes advantage of changes in tertiary structure induced by ligand binding (Soukup 1999). Sites of structural heterogeneity exhibit greater levels of spontaneous cleavage, and can be identified upon resolution of the cleavage products by polyacrylamide gel electrophoresis (PAGE). When radiolabeled 106 queC is analyzed in the presence of either 1 μM or 10 μM preQ₁, the resulting pattern of spontaneous cleavage products undergoes marked change in comparison to the cleavage product profile generated in the absence of ligand (FIG. 3c). As is the case with many riboswitches, the regions of the queC motif experiencing structure modulation correlate generally with phylogenetically conserved sequences. The similarity in the degree of RNA structure modulation observed using preQ₁ concentrations of both 1 μM and 10 μM suggests that the dissociation constant (K_{d}) for this interaction is less than 1 μM.

The preQ₁-dependent changes in spontaneous cleavage occur in putative loops or other non-helical elements, supporting their involvement in tertiary structure formation. In contrast, structural modulation is not observed in the immediate vicinity of the predicted intrinsic terminator, corroborating the notion that this stem is a component of the expression platform and does not contact the ligand directly. Among the 36 queC motifs in the phylogeny (FIG. 1), 22 appear to be controlled at the translational level and 14 at the transcriptional level. Of the riboswitches that contain terminators, compelling antiterminators can be identified in 10. The association of this riboswitch with genes involved in preQ₁ anabolism suggests that it serves to down-regulate gene expression in response to ligand binding.

d. Selective preQ₁ Recognition by the queC Motif

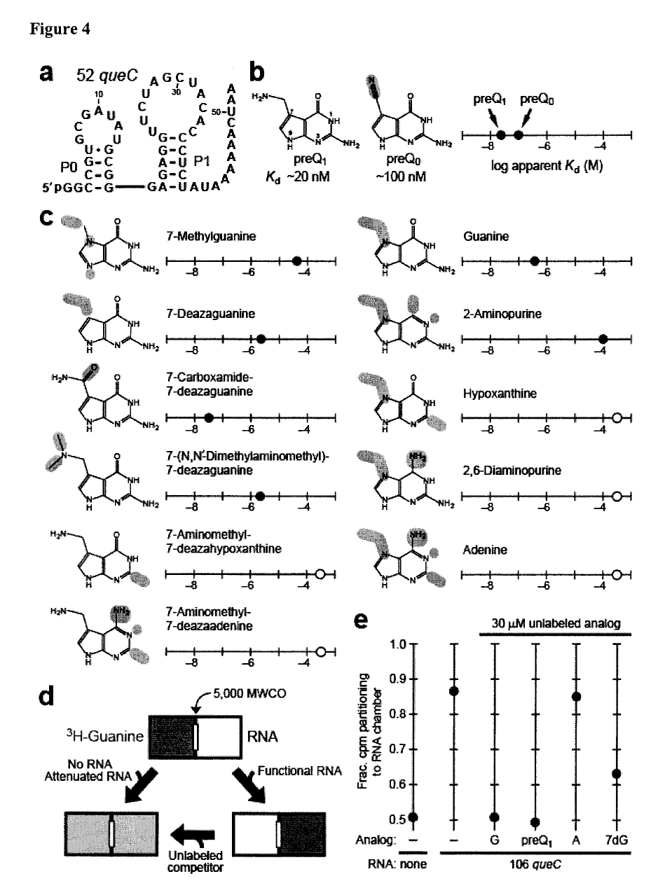

RNA construct 52 queC (FIG. 4a) was used to examine the degree of ligand selectivity of the B. subtilis queC element. Affinities for various purine compounds were determined by subjecting 5′³²P-labeled 52 queC to in-line probing analyses using a range of ligand concentrations, followed by the quantitative analysis of the levels of ligand-dependent structure modulation. PreQ₁ and the biosynthetic intermediate preQ₀ are recognized by 52 queC with K_{d} values of approximately 20 nM and 100 nM, respectively (FIG. 4b). These values suggest that preQ₁ is the primary target of the queC RNA element, and it seems reasonable from a gene-control perspective that the final Q biosynthetic intermediate that exists as a free nucleobase would serve as the regulator. Based only on the slight differential in affinity, however, a structurally related precursor like preQ₀ cannot be excluded as a physiologically relevant candidate ligand for this RNA motif.

The possibility that members of the two motif types (FIGS. 1 and 3a) might be selective for distinct yet related metabolites was examined. Having established that the type II queC element from B. subtilis preferentially binds preQ₁ (FIG. 4b), w the target selectivity of type I representatives was next examined. In-line probing assays with sequences corresponding to the two type I queC motifs identified in Bacillus cereus reveal ligand specificities identical to that of the type II example. These results indicate that preQ₁ rather than preQ₀ is likely to be the principal target of both types of the queC motif. In view of these results, the different L1 signature sequences that define these types can offer subtly different structural solutions for recognition of the same metabolite.

A more detailed understanding of the molecular contacts involved in ligand recognition by 52 queC was obtained by determining the apparent K_{d} values for a series of purine analogs (FIG. 4c). As expected, the chemical features that distinguish preQ₁ from guanine—the aminomethyl group and carbon atom substitution at the 7 position—are molecular recognition determinants for the RNA aptamer. The placement of two methyl groups at the nitrogen of the aminomethyl moiety [7-(N,N′-dimethylaminomethyl)-7-deazaguanine] decreases binding affinity substantially, as does the removal of the aminomethyl group (7-deazaguanine). In the former case, the weaker binding interaction relative to preQ₁ can result either from steric interference or from the absence of hydrogen bonding potential. The diminished binding affinity observed with 7-methylguanine, which lacks the carbon atom substitution, is even more pronounced, but this can result partly from the loss of a hydrogen bond donor at the N9 position. Interestingly, the apparent K_{d} for the amide analog of preQ₁, 7-carboxamide-7-deazaguanine is similar to that of preQ₁, indicating a degree of steric tolerance at the pertinent methylene group. Presumably, since the carboxamide derivative is not likely to be biologically relevant, no selective pressure exists for molecular discrimination against this compound.

Among the preQ₁ analogs that induce some degree of structure modulation with 52 queC, guanine alone is expected to accumulate under physiological conditions. The affinity of 52 queC for preQ₁ is only about 25-fold greater than for guanine, raising the question of whether this difference in ligand affinity is sufficient to account for the selective binding that presumably occurs in vivo. One factor that could contribute to higher selectivity toward preQ₁ in vivo is a limitation in the maximum cellular concentration of guanine. Also, several studies have shown that the kinetics of ligand association during transcription are critical to riboswitch function and that there is not necessarily an equivalence between K_{d} values and ligand concentrations to which riboswitches respond in vivo (Wickisier 2005a; Wickisier 2005b; Lemay 2006). It is therefore possible that the preQ₁-specific response of queC RNA can be aided by kinetic differences in ligand association between preQ₁ and guanine.

It is evident from the comparative binding affinity data that the exocylic amine at the 2 position of preQ₁ is a critical determinant of ligand recognition by queC RNA. Among the compounds that were tested, none that lacks this functional group induces structural modulation in queC RNA constructs, even when present at concentrations approaching 1 mM. The contribution of the 2-amino group to ligand binding can be examined in isolation by comparing the affinities of preQ₁ and 7-aminomethyl-7-deazahypoxanthine, whose only structural difference relative to preQ₁ is the absence of this functional group. Compared to the avid interaction of 52 queC with preQ₁, the affinity for 7-aminomethyl-7-deazahypoxanthine is reduced by at least four orders of magnitude, demonstrating that the 2-amino group is a major specificity determinant. This observation, combined with the adverse effects on ligand binding that result from the substitution or removal of a host of other functional groups of preQ₁, indicate that queC RNA serves as a highly selective aptamer for this modified base.

e. Assessing preQ₁ Recognition by Equilibrium Dialysis

To provide additional support for a specific physical interaction between preQ₁ and the queC RNA motif, competitive binding experiments were done using an equilibrium dialysis apparatus containing two chambers separated by a permeable membrane with a molecular weight cut-off of 5000 Daltons (FIG. 4d). Because guanine is recognized by the queC motif with nanomolar affinity (FIG. 4c), we first added ³H-guanine and a molar excess of 106 queC RNA individually to the respective chambers. As expected, the redistribution of tritium to the RNA-containing chamber following an equilibration period of 10 to 15 h (FIG. 4e) was observed. This sequestration of ³H-guanine can be competitively reversed by the addition of unlabeled guanine, preQ₁, or 7-deazaguanine when added at a concentration exceeding that of the RNA, while adenine did not serve as a competitor under these conditions (FIG. 4e). These results support the conclusion that queC RNA interacts selectively with preQ₁ and closely related purines.

f. Minimum Length for the preQ₁ Aptamer

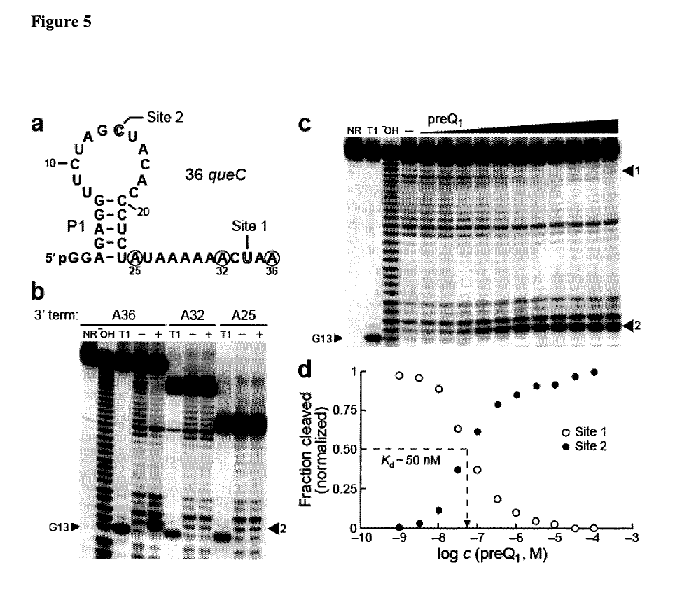

The poor phylogenetic conservation of the P0 stem-loop substructure (FIG. 1) shows that its role in aptamer function is not essential. To test this possibility, 36 queC was prepared, which is truncated at the 5′ end to eliminate P0 (FIG. 5a). In-line probing analysis using 5′³²P-labeled 36 queC reveals that, in the presence of 10 μM preQ₁, structure modulation occurs in regions within the loop and 3′ tail, indicating that the absence of P0 does not result in catastrophic loss of aptamer function (FIG. 5b).

To investigate whether conserved sequences near the 3′ end of the queC motif are required for ligand binding, two derivative deletion constructs with 3′ termini corresponding to A32 and A25 were formed. Neither of these truncated RNAs exhibits preQ₁-dependent structural changes, supporting the participation of the tail segment in ligand recognition (FIG. 5b). These data confirm that the relatively compact region of primary and secondary structure conservation that defines the queC motif contains all of the sequences necessary for selective recognition of preQ₁. Notably, binding experiments with synthetic RNAs provide evidence that the two 5′ guanosyl residues of 36 queC are dispensable for ligand binding, indicating that the length of the minimal preQ₁ aptamer in B. subtilis is only 34 nt.

To test whether the loss of P0 compromised the avidity of ligand binding, the K_{d} for preQ₁ was determined by subjecting 36 queC to in-line probing analysis in the presence of increasing preQ₁ concentrations (FIG. 5c). Quantitative analysis of structure modulation at two selected sites reveals a K_{d} of approximately 50 nM (FIG. 5d). A construct that contains P0 exhibits a slightly lower K_{d} value (20 nM; FIG. 4b), which suggests that this stem-loop makes only a small contribution to ligand binding. There is also the possibility that the presence of P0 could have an impact on kinetic aspects of riboswitch function. Nonetheless, it is apparent from these data that any enhancement of binding affinity attributable to P0 would not be substantial, and this conclusion is consistent with the limited phylogenetic distribution of this substructure.

g. Evidence for a Canonical Base Pair in preQ₁ Recognition

The selectivity of guanine riboswitches relies in part on the formation of a Watson-Crick base pair with its ligand (Gilbert 2006; Mandal 2004; Batey 2004). The cytidyl residue that base pairs with the ligand can be mutated to a uridyl residue to change the aptamer specificity from guanine to adenine (Gilbert 2006; Mandal 2004). This single point mutation permits a canonical base-pairing interaction with the adenine ligand without perturbing intermolecular contacts elsewhere. Because the structurally similar metabolites guanine and preQ₁ have the same capacity for canonical base-pair formation with cytidine, a search for a similar ligand-cytidine pairing interaction with preQ₁ aptamers was carried out. By analogy with the guanine riboswitch, it was reasoned that any cytidyl residue of queC RNA serving this hypothetical role would be absolutely conserved, and that a mutation to uridine at this position would be sufficient to switch ligand specificity to favor compounds more closely related to adenine.

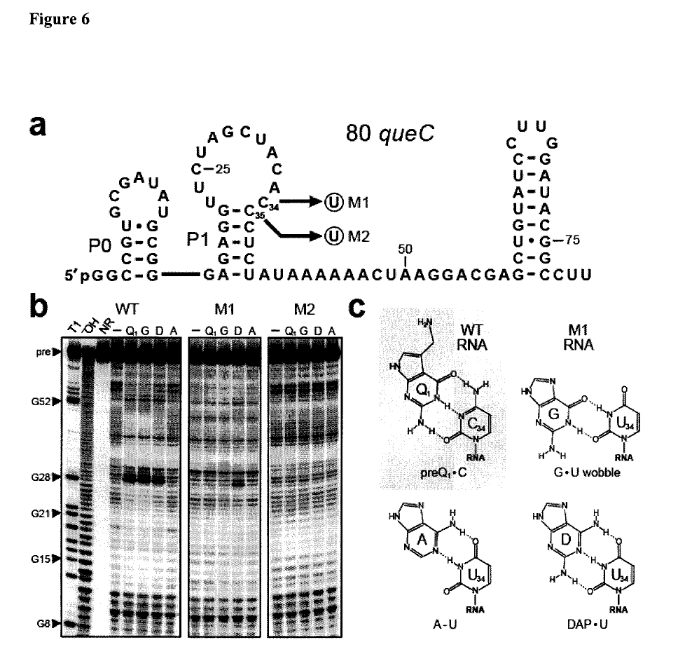

This possibility was examined by comparing the ligand binding characteristics of a wild-type queC construct (80 queC) with those of two mutant RNAs in which absolutely conserved cytidine residues were individually replaced with uridines (FIG. 6a). 2,6-diaminopurine was included in the panel of test compounds since it was previously established that the 2-amino group was critical for ligand recognition by the preQ₁ aptamer (FIG. 4c). It was therefore anticipated that compounds lacking this moiety, such as adenine or 7-aminomethyl-7-deazaadenine, might not bind detectably, despite their potential for base pairing with a uridyl discriminator residue.

In-line probing analysis using wild-type 80 queC demonstrates that structure modulation occurs in the presence of 1 μM preQ₁ or 200 μM guanine, as expected (FIG. 6b). Structural changes in this construct are also induced by 200 μM 2,6-diaminopurine, which is consistent with prior observations. Although the K_{d} for the interaction with 80 queC appears to exceed 300 μM (FIG. 4c), alterations in structure induced by 2,6-diaminopurine are detected in concentrations as low as 10 μM. The presence of adenine does not result in structure modulation in wild-type 80 queC even at a concentration of 200 μM.

The mutant version of 80 queC (M1) in which cytidine 34 is replaced with uridine (M1) displays a selectivity profile that differs markedly from that of the wild-type construct (FIG. 6b). No structure modulation is observed in M1 upon incubation with preQ₁ or guanine. Moreover, despite the failure of adenine to induce any conformational response, key changes are observed in M1 upon incubation with 2,6-diaminopurine. Importantly, these structural changes are similar to ones that are elicited by preQ₁ in the wild-type construct, with the increased levels of strand cleavage at internucleotide linkages immediately 3′ to C29 reflecting the most obvious among these (FIG. 6b). In contrast, none of the test compounds induces structural modulation in M2, a construct in which the other absolutely conserved cytidine (C35) is replaced by uridine.

These data show that, in wild-type 80 queC, C34 can form a Watson-Crick base pair with the preQ₁ ligand analogously to the discriminator cytidine of the guanine riboswitch (FIG. 6c). The absence of adenine binding by M1 is entirely consistent with previous observations that removal of the 2-amino group has a severely negative impact on ligand recognition (FIG. 4c). In fact, concentrations of 7-aminomethyl-7-deazaadenine as high as 1 mM also fail to modulate the structure of a queC RNA construct containing the analogous C34U mutation, emphasizing the impact of the absence of this moiety.

Despite the potential for formation of a non-canonical wobble pair between uridine 34 and either guanine or preQ₁, no binding of these nucleobase ligands to M1 RNA was observed. If uridine 34 indeed participates in a base-pairing interaction with 2,6-diaminopurine, then the exclusion of preQ₁ and guanine probably results from the spatial constraints imposed by the binding pocket. Aptamer contacts with the Hoogsteen and sugar edges of the ligand would be incompatible with the shift of approximately 2 Å that can be required for wobble pair formation (FIG. 6c).

Although the qualitative effect of 2,6-diaminopurine on the structure of M1 RNA is clear, it appears that the extent of this modulation is not as pronounced as that observed with wild-type RNA in response to preQ₁ or guanine (FIG. 6b). This apparent decrease in the degree of structure modulation presumably reflects an impairment in the ability of the mutant to bind 2,6-diaminopurine. Consistent with this, in-line probing methods revealed a K_{d} for this interaction of greater than 300 μM (data not shown). In comparison, the K_{d} for the interaction between wild-type queC RNA and guanine is approximately 500 nM (FIG. 4c), indicating that, even with an appropriately base-pairing ligand, the C34U mutant experiences a relative loss in binding affinity of more than 600 fold. Like 2,6-diaminopurine, guanine lacks the 7-deaza-7-aminomethyl modification, thereby permitting a direct comparison of the effects stemming only from the Watson-Crick edges of the ligand and the discriminator base.

Possible reasons for this disparity include adverse effects on global aptamer structure that are likely to result from substitutions of key functional groups. For example, the primary chemical difference resulting from the C34U mutation is the effective replacement of the exocyclic amine at the 4 position with a keto group (FIG. 6c). Although this functional group exchange allows for base pairing with 2,6-diaminopurine, it could nevertheless disrupt critical intramolecular contacts within the aptamer, thereby detracting from the stability of the overall fold. Similarly, it is possible that the 6-keto oxygen of preQ₁ may contact other molecular determinants in addition to the 4-amino group of C34.

h. The preQ₁ Riboswitch is a Gene Control Element

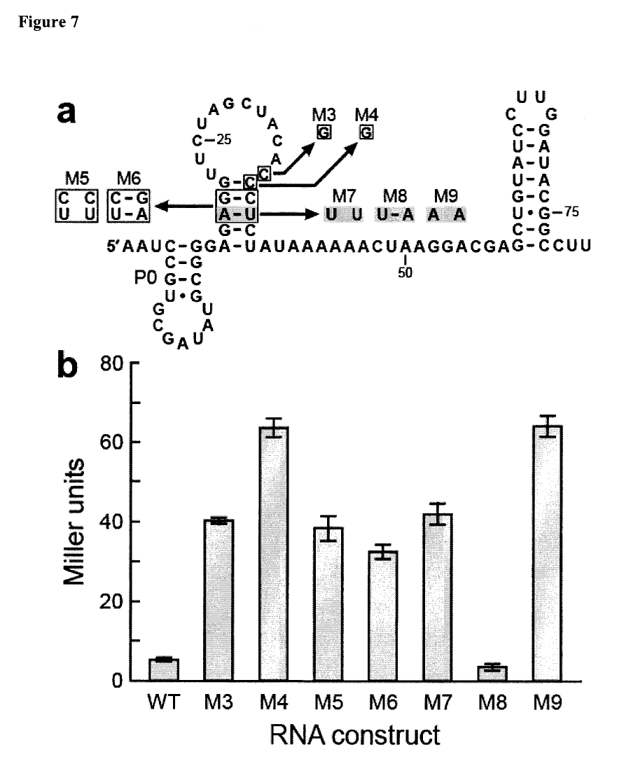

The recognition of preQ₁ with high affinity and selectivity by the queC motif, combined with the frequent association of this element with genes involved in preQ₁ biosynthesis, strongly suggest that representatives of this motif serve as ligand-binding components of riboswitches that control gene expression in response to preQ₁ levels. To test whether the queC element functions in this capacity, wild-type or engineered versions of 5′ UTR sequences from the B. subtilis queCDEF operon were joined to β-galactosidase reporter gene (FIG. 7a). This series of transcriptional fusion constructs was then used to generate B. subtilis chromosomal transformants, and individual strains were assayed for levels of β-galactosidase activity following growth in rich medium.

A transformant containing the lacZ gene coupled to a wild-type 5′ UTR sequence displayed relatively low levels of β-galactosidase activity when grown in the absence of preQ₁ supplementation. This result shows that the queC element acts to repress gene expression, most likely by responding to preQ₁ naturally produced by proteins expressed from the queCDEF operon (FIG. 7b). In contrast, B. subtilis strains harboring constructs in which key aptamer residues are mutated (M3 and M4) are derepressed. Because the mutations in M3 and M4 occur at positions demonstrated to be critical for the recognition of preQ₁ (FIGS. 6a,b), this result provides a direct correlation between aptamer function and genetic control.

Assays using a construct with a disruption of two by within the P1 stem (M5) also resulted in a loss of lacZ repression, although experiments with another variant, which contained compensatory mutations designed to restore stem integrity (M6), elicited a similar degree of derepression. The failure of M6 to repress lacZ expression analogously to the wild-type sequence is probably attributable to the strong bias in the nucleotide identities at these positions, which is revealed by the alignment of queC motif representatives (FIG. 1). Because no such bias is observed for the adjacent A:U base pair, a more focused mutational analysis of just these two nt was performed. The subset of mutants employed revealed that disruption of P1 structure correlates with lacZ derepression (M7 and M9), while a compensatory mutation (M8) that restores P1 integrity results in levels of β-galactosidase repression similar to those effected by wild type. Together, these data indicate that any interference with aptamer function resulting from mutation of phylogenetically conserved sequences or structures correlates with impairment of genetic control. These observations support the conclusion that the queC motif is the aptamer component of a preQ₁-sensing riboswitch.

ii. Discussion

The use of bioinformatics approaches to search for conserved nucleic acid motifs has uncovered several riboswitches as well as a number of riboswitch candidates (Barrick 2004; Corbino 2008; Fuchs 2006). The specific roles of many of these candidate motifs remain obscure, in part because the poorly understood functions of their associated genes present a barrier to further analysis. The queC motif, which was originally identified in conjunction with genes of unknown function (Barrick 2004), typified such a quandary. However, the discovery that the genes of the queCDEF operon are involved in Q biosynthesis (Reader 2004) provided key information in elucidating the function of this conserved motif as a preQ₁ riboswitch.

In reciprocal fashion, the identification of preQ₁ as the target metabolite of the queC element can help to shed light on the roles of other genes in this new-found regulon that remain uncharacterized. For example, the recurrent juxtaposition of a preQ₁ riboswitch with a gene encoding a predicted membrane protein (COG4708; FIG. 8) implies a role for this protein in the transport of Q or a related metabolite. In addition, preQ₁ riboswitches are associated in several instances with homologous operons containing two genes of unknown functions (FIG. 8). Based on COG database assignments (Tatusov 2001), one of these genes is predicted to encode a protein with similarity to inosine-uridine nucleoside N-ribohydrolase. Because enzymes of this class participate in nucleobase salvage by hydrolyzing the N-ribosidic bonds of nucleosides (Dewey 1973; Miller 1984), it is reasonable to speculate that the products of homologous genes associated with preQ₁ riboswitches catalyze the analogous reaction with Q or related substrates. The other gene in this putative bicistronic operon is predicted to encode a conserved membrane protein, which can function as a transporter. Given that their expression appears to be controlled by preQ₁ levels, these genes are likely to comprise a salvage operon involved in the transport and recycling of Q or its derivatives. In support of this hypothesis, specific enzyme systems have been identified in mammalian cells that act on queuosine 5′-phosphate to recover the queuine base following tRNA turnover (Gunduz 1982; Gunduz 1984).

One of the most striking qualities of the preQ₁ class of riboswitches is the small size of its aptamer. All of the determinants required for selective, high affinity target recognition in vitro can be contained within a span of only 34 nt. The features of this small conserved RNA element consist simply of a stem-loop and a short, 3′ tail carrying several consecutive adenosine residues. It is conceivable that A-minor interactions (Nissen 2001) between this adenosine-rich segment and the P1 minor groove can contribute to the tertiary structure of the aptamer in its ligand-bound state.

Despite its remarkably small size, the preQ₁ motif appears nonetheless to be capable of serving as an effective agent of gene control in a variety of eubacteria. This demonstrates clearly that large size and structural complexity are not required elements for the function in vivo of metabolite-binding RNAs. Furthermore, because it is presumed that comparatively small RNA motifs are generally less amenable to detection using automated searching methods, motifs similar in size to that of the preQ₁ aptamer can comprise a substantial fraction of metabolite-binding domains yet to be discovered.

In many respects, the capacity of a natural, miniature RNA for sophisticated function was presaged by the abundance of diminutive aptamer domains that have been isolated using in vitro selection. Although it is unusually small in comparison to natural examples, the size of the preQ₁ aptamer is unexceptional when measured against those that have been evolved in the laboratory. The preQ₁-binding motif is distinctive, however, in terms of the affinity and selectivity of its interaction with the cognate ligand. Artificially generated aptamers, whose lengths are constrained by the pools from which they are derived, are generally observed to bind their targets with poorer affinities and selectivities than naturally occurring motifs (Breaker 2006). The limitations of aptamers selected in vitro can be attributed to a combination of factors, including less stringent selection pressure and obstructed access to the ligand resulting from immobilization methods. It is apparent, however, that small size alone does not preclude RNA from specific, high affinity recognition of small molecule targets.

iii. Methods

Oligonucleotides and Chemicals. Synthetic DNA oligonucleotides were prepared using standard solid-phase methods by the HHMI Biopolymer/Keck Foundation Biotechnology Resource Laboratory. Following purification by denaturing (8 M urea) polyacrylamide gel electrophoresis (PAGE), oligonucleotides were eluted from gel fragments in 10 mM Tris-HCl (pH 7.5 at 23° C.), 200 mM NaCl and 1 mM EDTA, and subsequently concentrated by precipitation with ethanol. 7-deazahypoxanthine and 7-deazaadenine were obtained from Berry & Associates. Other purine compounds were purchased from Sigma-Aldrich.

PreQ₁ was synthesized as described (Akimoto 1988) and purified by reverse-phase HPLC (Luna C18, 250×10 mm, 5 μm, (Phenomenex)) with a flow rate of 5 mL min⁻¹ using an isocratic mobile phase of 20 mM ammonium acetate (pH 6.0). PreQ₁ eluted at 17 min. Fractions containing preQ₁ were collected, frozen and lyophilized to yield the product as a white powder. ¹H-NMR (D₂O) δ 6.88 (s, 1H, C—H), 4.13 (s, 2H, CH₂), 1.95 (s, 3H, acetate salt).

7-(N,N′-dimethylaminomethyl)-7-deazaguanine, the dimethyl analog of preQ₁, was synthesized as described (Akimoto 1988) and recrystallized from EtOH to yield the product as a white solid. ¹H-NMR (d₆-DMSO) δ 10.8 (s, 1H, N—H) 10.2 (br s, 1H, N—H), 6.44 (s, 1H, C—H), 6.00 (s, 2H, NH₂), 3.51 (s, 2H, CH₂), 2.13 (s, 6H, 2×CH₃).

7-cyano-7-deazaguanine, or preQ₀, was synthesized as described (Migawa 1996) and purified by reverse-phase HPLC (Luna C18, 250×10 mm, 5 (Phenomenex)) with a flow rate of 5 mL min⁻¹ using an isocratic mobile phase of 4% (v/v) acetonitrile in 20 mM ammonium acetate (pH 6.0). PreQ₀ eluted at 15 min, and lyophilization of the relevant fractions afforded preQ₀ as an off-white solid. ¹H-NMR (d₆-DMSO) δ 11.97 (s, 1H, N—H), 10.70 (s, 1H, N—H), 7.61 (s, 1H, C—H), 6.38 (s, 2H, NH₂).

7-carboxamide-7-deazaguanine, the amide analog of preQ₁, was synthesized as described (Migawa 1996) and recrystallized from EtOH to yield the product as a yellow powder. ¹H-NMR (d₆-DMSO) δ 11.60 (br s, 1H, NH), 10.85 (br s, 1H, NH), 9.55 and 6.95 (2s, 2H, CONH₂), 7.22 (s, 1H, C—H), 6.40 (s, 2H, NH₂).

7-aminomethyl-7-deazahypoxanthine was prepared using a modified version of a previously reported protocol (Akimoto 1986). The intermediate, 7-(N,N′-dimethylaminomethyl)-7-deazahypoxanthine was synthesized according to previously published procedures⁵⁰. ¹H NMR (DMSO, 400 MHz) δ9.83 (br s, 1H, 4-OH), 7.58 (s, 1H, 8-NH), 7.43 (s, 1H, 2-H), 4.13 (s, 2H, 5-CH₂NH₂), 2.51 (s, 2H, 5-CH₂NH₂); ¹³C NMR (DMSO, 100 MHz) δ132.4, 130.5, 129.2, 128.9, 82.1, 69.5, 50.1.

7-aminomethyl-7-deazaadenine was prepared using a modified version of a previously reported protocol Akimoto 1986). The intermediate, 7-(N,N′-dimethylaminomethyl)-7-deazaadenine was synthesized following previously published procedures (GB Patent No. 981458 (1965)). R_{f} 0.33 (MeOH:CH₂Cl₂=9:1); mp ° C.; ¹H NMR (DMSO, 500 MHz) δ11.21 (s, 2H, 4-NH₂), 7.79 (s, 1H, 7-NH), 6.83 (s, 1H, 2-H), 6.65 (s, 2H, 5-CH₂NH₂), 6.28 (s, 1H, 8-H), 1.69 (s, 2H, 5-CH₂NH₂); ¹³C NMR (DMSO, 125 MHz) δ 157.7, 151.9, 150.8, 140.8, 121.2, 120.6, 99.2; IR (neat, cm⁻¹); MS (EI) m/e 163 (M⁺).

Bioinformatics. PreQ₁ aptamer sequence alignments were manually adapted to the secondary structure model presented in this report from a previously published alignment of the ykvJ RNA motif (Barrick 2004). Covariance models (Eddy 1994) trained on this initial alignment were used to search microbial genomes and environmental sequences for additional matches with the RAVENNA extension (Weinberg 2006) (which accelerates covariance model searches with sequence-based heuristic filters) to the INFERNAL software package (Eddy 2003). PreQ₁ riboswitch candidates were verified by examining their genomic contexts, which involved using the COG database (Tatusov 2003) to predict the functions of genes in putative operons. Sequences in the final alignment were weighted with the GSC algorithm (Gerstein 1994) to mitigate biases from similar sequences before calculating the reported consensus sequence.

RNA construct preparation. A portion of the intergenic region upstream of queC was amplified using PCR from B. subtilis genomic DNA (strain 1A40) using the primers 5′-GAGCCTGGAATTCATAGGCGCTTTGC (SEQ ID NO: 1) and 5′-TTTTCTGGATCCATGATTCCTC-TCC (SEQ ID NO: 2). Following digestion with EcoRI and BamHI, the amplification product was cloned into pDG1661 (ref. 57) and the integrity of the resulting plasmid was confirmed by sequencing. This plasmid served as the template for PCR amplification of the DNA fragment encoding the 106 queC construct. DNA templates corresponding to the remaining RNA constructs were prepared by extending appropriate partially complementary synthetic oligonucleotides using SuperScript II reverse transcriptase (Invitrogen) according to the manufacturer's instructions. For each oligonucleotide pair used in the production of transcription templates, the primer in the sense orientation contained the T7 promoter sequence. RNA molecules were prepared by transcription in vitro using T7 RNA polymerase and gel-purified as previously described (Roth 2006).

In-line probing analysis. Enzymatically synthesized RNA molecules were dephosphorylated with alkaline phosphatase (Roche Diagnostics) and radiolabeled with γ-³²P [ATP] and T4 polynucleotide kinase (New England Biolabs) according to the manufacturers' instructions. Spontaneous transesterification reactions using gel-purified, 5′ end-labeled RNAs were assembled essentially as previously described (Mandal 2003). Incubations were approximately 40 h at 25° C. in 10 μL volumes containing 50 mM Tris-HCl (pH 8.3 at 23° C.), 20 mM MgCl₂, 100 mM KCl, and ˜5 nM precursor RNA in the presence or absence of test compounds as indicated for each experiment. PreQ₁ and related compounds were typically tested at concentrations ranging from 10 nM to 200 μM, although certain compounds with weaker binding affinities were assayed at concentrations exceeding this range. RNA fragments resulting from spontaneous transesterification were resolved by denaturing 10% PAGE, and the imaging and quantitation of these data were performed with a Molecular Dynamics PhosphorImager and ImageQuaNT software. K_{d} values were determined as described previously (Mandal 2003).

Equilibrium dialysis. Samples containing either 100 nM ³H-guanine or 20 μM 106 queC RNA, each prepared in the same buffer solution used for in-line probing, were added in 30 μL volumes to opposite chambers of a DispoEquilibrium Dialyzer (ED-1, Harvard Bioscience). Following equilibration at 25° C. for 10-15 h, aliquots of 5 μL were withdrawn and quantitated with a liquid scintillation counter. For competitive binding experiments, 3 μL of buffer containing 1 mM unlabeled test compound was subsequently delivered to the RNA-containing chamber, while the same volume of buffer was added to the opposite chamber. After another equilibration period, 5 μL aliquots were again removed to assess the distribution of ³H-guanine.

Analysis of preQ₁ riboswitch function in vivo. The function in vivo of the preQ₁ riboswitch was assessed by fusing sequences containing this element with a lacZ reporter gene using methods similar to those described previously⁶⁰. A portion of the intergenic region upstream of queC was amplified using PCR from B. subtilis genomic DNA (strain 1A40) using the primers 5′-CGAGAATTCATAATGAAACGAACCGTCACTATAG (SEQ ID NO: 3) and 5′-GTACTTTTTTCTTTTTCGTTAACAGCCTAGGTGC (SEQ ID NO: 4). Following digestion with EcoRI and BamHI, the amplification product was cloned into pDG1661 immediately upstream of lacZ and the integrity of the resulting plasmid was confirmed by sequencing. To generate sequence variants M3-M9, site-directed mutagenesis of the wild-type construct was performed using a QuikChange kit (Stratagene) together with primers that carried the desired mutations. Constructs were integrated at the amyE locus in strain 1A40 and confirmed as described (Mandal 2003). Following growth in 2XYT broth with shaking at 37° C. to an A₆₀₀ of 0.6, these strains were used in β-galactosidase assays.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a riboswitch” includes a plurality of such riboswitches, reference to “the riboswitch” is a reference to one or more riboswitches and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Ptashne, M. & Gann, A. Genes and Signals (Cold Spring Harbor Laboratory Press, Cold Spring Harbor, N.Y., 2002).
2. Mandal, M. & Breaker, R. R. Gene regulation by riboswitches. Nat. Rev. Mol. Cell. Biol. 5, 451-463 (2004).
3. Soukup, J. K. & Soukup, G. A. Riboswitches exert genetic control through metabolite-induced conformational change. Curr. Opin. Struct. Biol. 14, 344-349 (2004).
4. Winkler, W. C. Riboswitches and the role of noncoding RNAs in bacterial metabolic control. Curr. Opin. Chem. Biol. 9, 594-602 (2005).
5. Winkler, W. C. & Breaker, R. R. Regulation of bacterial gene expression by riboswitches. Annu. Rev. Microbiol. 59, 487-517 (2005).
6. Breaker, R. R. in The RNA World, third edition (eds. Gesteland, R. F., Cech, T. R. & Atkins, J. F.) (Cold Spring Harbor Laboratory Press, Cold Spring Harbor, N.Y., 2006).
7. Barrick, J. E. et al. New RNA motifs suggest an expanded scope for riboswitches in bacterial genetic control. Proc. Natl. Acad. Sci. USA 101, 6421-6426 (2004).
8. Corbino, K. A. et al. Evidence for a second class of S-adenosylmethionine riboswitches and other regulatory RNA motifs in alpha-proteobacteria. Genome Biol. 6, R70 (2005).
9. Reader, J. S., Metzgar, D., Schimmel, P. & de Crécy-Lagard, V. Identification of four genes necessary for biosynthesis of the modified nucleoside queuosine. J. Biol. Chem. 279, 6280-6285 (2004).
10. Harada, F. & Nishimura, S. Possible anticodon sequences of tRNA^{His}, tRNA^{Asn}, and tRNA^{Asp} from Escherichia coli B. Universal presence of nucleoside Q in the first position of the anticodons of these transfer ribonucleic acids. Biochemistry 11, 301-308 (1972).
11. Van Lanen, S. G. et al. From cyclohydrolase to oxidoreductase: discovery of nitrile reductase activity in a common fold. Proc. Natl. Acad. Sci. USA 102, 4264-4269 (2005).
12. Gaur, R. & Varshney, U. Genetic analysis identifies a function for the queC (ybaX) gene product at an initial step in the queuosine biosynthetic pathway in Escherichia coli. J. Bacteriol. 187, 6893-6901 (2005).
13. Okada, N. et al. Novel mechanism of post-transcriptional modification of tRNA. Insertion of bases of Q precursors into tRNA by a specific tRNA transglycosylase reaction. J. Biol. Chem. 254, 3067-3073 (1979).
14. Reuter, K., Slany, R., Ullrich, F. & Kersten, H. Structure and organization of Escherichia coli genes involved in biosynthesis of the deazaguanine derivative queuine, a nutrient factor for eukaryotes. J. Bacteriol. 173, 2256-2264 (1991).
15. Salazar, J. C., Ambrogelly, A., Crain, P. F., McCloskey, J. A. & Soll, D. A truncated aminoacyl-tRNA synthetase modifies RNA. Proc. Natl. Acad. Sci. USA 101, 7536-7541 (2004).
16. Blaise, M. et al. A minimalist glutamyl-tRNA synthetase dedicated to aminoacylation of the tRNA^{Asp} QUC anticodon. Nucleic Acids Res. 32, 2768-2775 (2004).
17. Iwata-Reuyl, D. Biosynthesis of the 7-deazaguanosine hypermodified nucleosides of transfer RNA. Bioorg. Chem. 31, 24-43 (2003).
18. Durand, J. M. et al. vacC, a virulence-associated chromosomal locus of Shigella flexneri, is homologous to tgt, a gene encoding tRNA-guanine transglycosylase (Tgt) of Escherichia coli K-12. J. Bacteriol. 176, 4627-4634 (1994).
19. Noguchi, S., Nishimura, Y., Hirota, Y. & Nishimura, S. Isolation and characterization of an Escherichia coli mutant lacking tRNA-guanine transglycosylase. Function and biosynthesis of queuosine in tRNA. J. Biol. Chem. 257, 6544-6550 (1982).
20. Frey, B., Janel, G., Michelsen, U. & Kersten, H. Mutations in the Escherichia coli fnr and tgt genes: control of molybdate reductase activity and the cytochrome d complex byfnr. I Bacteriol. 171, 1524-1530 (1989).
21. Bienz, M. & Kubli, E. Wild type tRNA^{Tyr} reads the TMV RNA stop codon, but Q base-modified tRNA^{Tyr} does not. Nature 294, 188-190 (1981).
22. Meier, F., Suter, B., Grosjean, H., Keith, G. & Kubli, E. Queuosine modification of the wobble base in tRNA^{His} influences ‘in vivo’ decoding properties. EMBO J. 4, 823-827 (1985).
23. Urbonavi\check{c}ius, J., Qian, Q., Durand, J. M., Hagervall, T. G. & Björk, G. R. Improvement of reading frame maintenance is a common function for several tRNA modifications. EMBO J. 20, 4863-4873 (2001).
24. Kuchino, Y., Kasai, H., Nihei, K. & Nishimura, S. Biosynthesis of the modified nucleoside Q in transfer RNA. Nucleic Acids Res. 3, 393-398 (1976).
25. Okada, N. et al. Structure determination of a nucleoside Q precursor isolated from E. coli tRNA: 7-(aminomethyl)-7-deazaguanosine. Nucleic Acids Res. 5, 2289-2296 (1978).
26. Slany, R. K., Bosl, M., Crain, P. F. & Kersten, H. A new function of S-adenosylmethionine: the ribosyl moiety of AdoMet is the precursor of the cyclopentenediol moiety of the tRNA wobble base queuine. Biochemistry 32, 7811-7817 (1993).
27. Slany, R. K., Bosl, M. & Kersten, H. Transfer and isomerization of the ribose moiety of AdoMet during the biosynthesis of queuosine tRNAs, a new unique reaction catalyzed by the QueA protein from Escherichia coli. Biochimie 76, 389-393 (1994).
28. Frey, B., McCloskey, J., Kersten, W. & Kersten, H. New function of vitamin B₁₂: cobamide-dependent reduction of epoxyqueuosine to queuosine in tRNAs of Escherichia coli and Salmonella typhimurium. J. Bacteriol. 170, 2078-2082 (1988).
29. Gusarov, I. & Nudler, E. The mechanism of intrinsic transcription termination. Mol. Cell. 3, 495-504 (1999).
30. Yarnell, W. S. & Roberts, J. W. Mechanism of intrinsic transcription termination and antitermination. Science 284, 611-615 (1999).
31. Soukup, G. A. & Breaker, R. R. Relationship between internucleotide linkage geometry and the stability of RNA. RNA 5, 1308-1325 (1999).
32. Wickiser, J. K., Winkler, W. C., Breaker, R. R. & Crothers, D. M. The speed of RNA transcription and metabolite binding kinetics operate an FMN riboswitch. Mol. Cell. 18, 49-60 (2005).
33. Wickiser, J. K., Cheah, M. T., Breaker, R. R. & Crothers, D. M. The kinetics of ligand binding by an adenine-sensing riboswitch. Biochemistry 44, 13404-13414 (2005).
34. Lemay, J. F., Penedo, J. C., Tremblay, R., Lilley, D. M. & Lafontaine, D. A. Folding of the adenine riboswitch. Chem. Biol. 13, 857-868 (2006).
35. Gilbert, S. D., Stoddard, C. D., Wise, S. J. & Batey, R. T. Thermodynamic and kinetic characterization of ligand binding to the purine riboswitch aptamer domain. J. Mol. Biol. 359, 754-768 (2006).
36. Mandal, M. & Breaker, R. R. Adenine riboswitches and gene activation by disruption of a transcription terminator. Nat. Struct. Mol. Biol. 11, 29-35 (2004).
37. Batey, R. T., Gilbert, S. D. & Montange, R. K. Structure of a natural guanine-responsive riboswitch complexed with the metabolite hypoxanthine. Nature 432, 411-415 (2004).
38. Serganov, A. et al. Structural basis for discriminative regulation of gene expression by adenine- and guanine-sensing mRNAs. Chem. Biol. 11, 1729-1741 (2004).
39. Noeske, J. et al. An intermolecular base triple as the basis of ligand specificity and affinity in the guanine- and adenine-sensing riboswitch RNAs. Proc. Natl. Acad. Sci. USA 102, 1372-1377 (2005).
40. Fuchs, R. T., Grundy, F. J. & Henkin, T. M. The S(MK) box is a new SAM-binding RNA for translational regulation of SAM synthetase. Nat. Struct. Mol. Biol. 13, 226-233 (2006).
41. Tatusov, R. L. et al. The COG database: new developments in phylogenetic classification of proteins from complete genomes. Nucleic Acids Res. 29, 22-28 (2001).
42. Dewey, V. & Kidder, G. W. Partial purification and properties of a nucleoside hydrolase from Crithidia. Arch. Biochem. Biophys. 157, 380-387 (1973).
43. Miller, R. L., Sabourin, C. L., Krenitsky, T. A., Berens, R. L. & Man, J. J. Nucleoside hydrolases from Trypanosoma cruzi. J. Biol. Chem. 259, 5073-5077 (1984).
44. Gündüz, U. & Katze, J. R. Salvage of the nucleic acid base queuine from queuine-containing tRNA by animal cells. Biochem. Biophys. Res. Commun. 109, 159-167 (1982).
45. Gündüz, U. & Katze, J. R. Queuine salvage in mammalian cells. Evidence that queuine is generated from queuosine 5′-phosphate. J. Biol. Chem. 259, 1110-1113 (1984).
46. Nissen, P., Ippolito, J. A., Ban, N., Moore, P. B. & Steitz, T. A. RNA tertiary interactions in the large ribosomal subunit: the A-minor motif Proc. Natl. Acad. Sci. USA 98, 4899-4903 (2001).
47. Akimoto, H., Imamiya, E., Hitaka, T., Nomura, H. & Nishimura, S. Synthesis of queuine, the base of naturally occurring hypermodified nucleoside (queuosine), and its analogues. J. Chem. Soc. Perkin Trans. 1, 1637-1644 (1988).
48. Migawa, M. T., Hinkley, J. M., Hoops, G. C. & Townsend, L. B. A two step synthesis of the nucleoside Q precursor 2-amino-5-cyanopyrrolo[2,3-d]pyrimidine-4-one (preQ₀). Synth. Commun. 26, 3317-3322 (1996).
49. Akimoto, H. et al. Queuine analogues. Their synthesis and inhibition of growth of mouse L5178Y cells in vitro. J. Med. Chem. 29, 1749-1753 (1986).
50. West, R. A. 4-Hydroxypyrrolo[2,3-d]pyrimidine: Mannich reaction. J. Org. Chem. 26, 4959-4961 (1961).
51. Great Britain patent number 981458 (1965).
52. Eddy, S. R. & Durbin, R. RNA sequence analysis using covariance models. Nucleic Acids Res. 22, 2079-2088 (1994).
53. Weinberg, Z. & Ruzzo, W. L. Sequence-based heuristics for faster annotation of non-coding RNA families. Bioinformatics 22, 35-39 (2006).
54. Eddy, S. R. INFERNAL. Version 0.55. Distributed by the author. Dept. of Genetics, Washington University School of Medicine. St. Louis, Mo. (2003).
55. Tatusov, R. L. et al. The COG database: an updated version includes eukaryotes. BMC Bioinformatics 4, 41 (2003).
56. Gerstein, M., Sonnhammer, E. L. & Chothia, C. Volume changes in protein evolution. J. Mol. Biol. 236, 1067-1078 (1994).
57. Guérout-Fleury, A. M., Frandsen, N. & Stragier, P. Plasmids for ectopic integration in Bacillus subtilis. Gene 180, 57-61 (1996).
58. Roth, A., Nahvi, A., Lee, M., Jona, I. & Breaker, R. R. Characteristics of the glmS ribozyme suggest only structural roles for divalent metal ions. RNA 12, 607-619 (2006).
59. Mandal, M., Boese, B., Barrick, J. E., Winkler, W. C. & Breaker, R. R. Riboswitches control fundamental biochemical pathways in Bacillus subtilis and other bacteria. Cell 113, 577-586 (2003).
60. Sudarsan, N., Wickiser, J. K., Nakamura, S., Ebert, M. S. & Breaker, R. R. An mRNA structure in bacteria that controls gene expression by binding lysine. Genes Dev. 17, 2688-2697 (2003).

We claim:

1. A regulatable gene expression construct comprising
a nucleic acid molecule encoding an RNA comprising a preQ₁-responsive riboswitch operably linked to a coding region, wherein the riboswitch regulates expression of the RNA, wherein the riboswitch and coding region are heterologous.

2. The construct of claim 1 wherein the riboswitch comprises an aptamer domain and an expression platform domain, wherein the aptamer domain and the expression platform domain are heterologous.

3. The construct of claim 1 wherein the riboswitch comprises two or more aptamer domains and an expression platform domain, wherein at least one of the aptamer domains and the expression platform domain are heterologous.

4. The construct of claim 3 wherein at least two of the aptamer domains exhibit cooperative binding.

5. A riboswitch, wherein the riboswitch is a non-natural derivative of a naturally-occurring preQ₁-responsive riboswitch.

6. The riboswitch of claim 5 wherein the riboswitch comprises an aptamer domain and an expression platform domain, wherein the aptamer domain and the expression platform domain are heterologous.

7. The riboswitch of claim 6 wherein the riboswitch further comprises one or more additional aptamer domains.

8. The riboswitch of claim 7 wherein at least two of the aptamer domains exhibit cooperative binding.

9. The riboswitch of claim 5 wherein the riboswitch is activated by a trigger molecule, wherein the riboswitch produces a signal when activated by the trigger molecule.

10. A method of detecting a compound of interest, the method comprising
bringing into contact a sample and a riboswitch, wherein the riboswitch is activated by the compound of interest, wherein the riboswitch produces a signal when activated by the compound of interest, wherein the riboswitch produces a signal when the sample contains the compound of interest, wherein the riboswitch comprises a preQ₁-responsive riboswitch or a derivative of a preQ₁-responsive riboswitch.

11. The method of claim 10 wherein the riboswitch changes conformation when activated by the compound of interest, wherein the change in conformation produces a signal via a conformation dependent label.

12. The method of claim 10 wherein the riboswitch changes conformation when activated by the compound of interest, wherein the change in conformation causes a change in expression of an RNA linked to the riboswitch, wherein the change in expression produces a signal.

13. The method of claim 12 wherein the signal is produced by a reporter protein expressed from the RNA linked to the riboswitch.

14. A method comprising
(a) testing a compound for inhibition of gene expression of a gene encoding an RNA comprising a riboswitch, wherein the inhibition is via the riboswitch, wherein the riboswitch comprises a preQ₁-responsive riboswitch or a derivative of a preQ₁-responsive riboswitch,
(b) inhibiting gene expression by bringing into contact a cell and a compound that inhibited gene expression in step (a),
wherein the cell comprises a gene encoding an RNA comprising a riboswitch, wherein the compound inhibits expression of the gene by binding to the riboswitch.

15. A method of identifying preQ₁-responsive riboswitches, the method comprising assessing in-line spontaneous cleavage of an RNA molecule in the presence and absence of preQ₁, wherein the RNA molecule is encoded by a gene regulated by preQ₁,
wherein a change in the pattern of in-line spontaneous cleavage of the RNA molecule indicates a preQ₁-responsive riboswitch.

16. A method of inhibiting gene expression, the method comprising
(a) bringing into contact a compound and a cell,
(b) wherein the compound has the structure of Formula I: 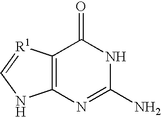
where R¹ is CH, N, C—NH₂, C—CH₂—NH₂, C—CN, C—C(O)NH₂, C—CH═NH, C—CH₂—N(CH₃)₂, or C-hydrogen bond donor,
wherein the cell comprises a gene encoding an RNA comprising a preQ₁-responsive riboswitch, wherein the compound inhibits expression of the gene by binding to the preQ₁-responsive riboswitch.

17. The method of any of claim 16, wherein the cell has been identified as being in need of inhibited gene expression.

18. The method of claim 16, wherein the cell is a bacterial cell.

19. The method of claim 18, wherein the compound kills or inhibits the growth of the bacterial cell.

20. The method of claim 16, wherein the compound and the cell are brought into contact by administering the compound to a subject.

21. The method of claim 20, wherein the cell is a bacterial cell in the subject, wherein the compound kills or inhibits the growth of the bacterial cell.

22. The method of claim 21, wherein the subject has a bacterial infection.

23. The method of any of claim 22, wherein the cell contains a preQ₁-responsive riboswitch.

24. The method of claim 16, wherein the compound is administered in combination with another antimicrobial compound.

25. The method of claim 16, wherein the compound inhibits bacterial growth in a biofilm.

26. A method of producing preQ₁, the method comprising:
(a) cultivating a mutant bacterial cell capable of producing preQ₁, wherein the mutant bacterial cell comprises a mutation in the preQ₁ riboswitch, which mutation increases preQ₁ production by the mutant bacterial cell in comparison to a cell not having the mutation;
(b) isolating preQ₁ from the cell culture, thereby producing preQ₁.

27. The method of claim 26, which method yields at least a 10% increase in preQ₁ production compared to cultivating a bacterial cell that does not comprise the mutation in the preQ₁ riboswitch.

28. The method of claim 26, which method yields at least a 10% increase in preQ₁ production compared to cultivating a bacterial cell that does not comprise the mutation in the preQ₁ riboswitch.

29. The method of claim 26, which method yields at least a 25% increase in preQ₁ production compared to cultivating a bacterial cell that does not comprise the mutation in the preQ₁ riboswitch.

30. The method of claim 26, wherein the mutation in the preQ₁ riboswitch is a knockout mutation.

31. A bacterial cell comprising a mutation in a preQ₁ riboswitch, which mutation measurably increases preQ₁ production by the cell when compared to a cell that does not have the mutation.

32. A method of inhibiting bacterial cell growth, the method comprising: bringing into contact a cell and a compound that binds a preQ₁-responsive riboswitch, wherein the cell comprises a gene encoding an RNA comprising a preQ₁-responsive riboswitch, wherein the compound inhibits bacterial cell growth by binding to the preQ₁-responsive riboswitch, thereby limiting preQ₁ production.

33. The method of claim 32, which method yields at least a 10% decrease in bacterial cell growth compared to a cell that is not in contact with the compound.

34. The method of claim 32, wherein the compound and the cell are brought into contact by administering the compound to a subject.

35. The method of claim 33, wherein the cell is a bacterial cell in the subject, wherein the compound kills or inhibits the growth of the bacterial cell.

36. The method of claim 33, wherein the subject has a bacterial infection.

37. The method of claim 32, wherein the compound is administered in combination with another antimicrobial compound.

38. A method of detecting preQ₁ in a sample comprising:
a. bringing a preQ₁-responsive riboswitch in contact with the sample; and
b. detecting interaction between preQ₁ and the preQ₁-responsive riboswitch, wherein interaction between preQ₁ and the preQ₁-responsive riboswitch indicates the presence of preQ₁.

39. The method of claim 38, wherein the preQ₁-responsive riboswitch is labeled.

40. A method comprising inhibiting gene expression of a gene encoding an RNA comprising a riboswitch by bringing into contact a cell and a compound that was identified as a compound that inhibits gene expression of the gene by testing the compound for inhibition of gene expression of the gene, wherein the inhibition was via the riboswitch, wherein the riboswitch comprises a preQ₁-responsive riboswitch or a derivative of a preQ₁-responsive riboswitch
